(19) United States
(12) Patent Application Publication
(10) Pub. No.: US 2023/0183807 A1
(43) Pub. Date: Jun. 15, 2023

(54) METHYLATION STATUS OF GASDERMIN E GENE AS CANCER BIOMARKER

(71) Applicants: UNIVERSITAIR ZIEKENHUIS ANTWERPEN, Edegem (BE); Universiteit Antwerpen, Antwerpen (BE)

(72) Inventors: Joe Ibrahim, Antwerpen (BE); Ken Op de Beeck, Kontich (BE); Arvid Suls, Mortsel (BE); Guido Van Camp, Duffel (BE); Marc Peeters, Waasmunster (BE)

(21) Appl. No.: 17/436,485

(22) PCT Filed: Mar. 4, 2020
(86) PCT No.: PCT/EP2020/055656

§ 371 Date: Sep. 3, 2021

(30) Foreign Application Priority Data
Mar. 4, 2019 (EP) 19160561.7
Nov. 13, 2019 (EP) 19208859.9

(51) Int. Cl. C12Q 1/6886

(52) U.S. Cl. CPC C12Q 1/6886; C12Q 2600/112; C12Q 2600/154

(57) ABSTRACT

The present invention applies to the area of cancer diagnostics. In particular, the present invention is directed to a method for the ex vivo differential diagnosis between several cancer types in a subject based on the methylation status of the Gasdermin E (GSDME) gene. In a further aspect, the present invention relates to a method for the ex vivo differential diagnosis between several cancer types based on the methylation status of at least 2 CpG sites in the GSDME gene.

### CROSS REFERENCES TO RELATED APPLICATIONS

This application is a national-stage application under 35 U.S.C. § 371 of International Application No. PCT/EP2020/0555656, filed Mar. 4, 2020, which claims priority to European Patent Application EP 191605617.7, filed Mar. 4, 2019, and to European Patent Application EP 192088599.9, filed Nov. 13, 2019.

### TECHNICAL FIELD

The present invention applies to the area of cancer diagnostics. In particular, the present invention is directed to a method for the ex vivo differential diagnosis between several cancer types in a subject based on the methylation status of the Gasdermin E (GSDME) gene. In a further aspect, the present invention relates to a method for the ex vivo differential diagnosis between several cancer types based on the methylation status of at least 2 CpG sites in the GSDME gene.

### BACKGROUND

Cancer is the second leading cause of death worldwide with 9.6 million deaths and 17 million new cases occurring yearly. The five most prevalent cancers worldwide include lung, breast, colorectal, prostate and gastric cancer. Despite advances in diagnosis and treatment, the socio-economic burden of cancer still weighs heavily on societies worldwide. Novel, accurate and cost-effective diagnostic strategies are needed for improved treatment and optimal disease management. In recent years, the use of biologically identifiable characteristics, more commonly known as biomarkers, to indicate the presence of cancer in the body has gained considerable attention. Studies have examined several sources of biomarkers, including DNA mutations, metabolites, gene and protein expression, mRNA, imaging and antibodies amongst others.

More recently epigenetic alterations, most notably DNA methylation, have garnered much attention in the context of putative cancer markers for diagnosis and early detection. In brief, DNA methylation is the addition of a methyl group predominantly to cytosine bases on the DNA backbone. Aberrant DNA methylation patterns are considered a hallmark of cancer (Kulis and Esteller, 2010). Several studies have demonstrated the repression of tumour suppressor genes involved in cellular signaling pathways, via promoter hypermethylation. Global genomic hypomethylation has also been associated with genomic-instability and silenced gene re-expression. Various studies have already outlined the potential of methylation as a biomarker for the early detection, diagnosis and prognosis of cancer. Only four commercially available DNA methylation analytical kits for cancer diagnosis currently exist. These use the genes VIM (cologuard) and SEPT9 (Epi proColon, ColoVantage and RealTime mS9) for colorectal cancer, SHOX2 (Epi prolong) in lung cancer and GSTP1/APC/RASSF1A (ConfirmMDx) in prostate cancer. These assays however, demonstrate a varying performance across tumour stages and are often ineffective at detecting residual disease. More recently, (Cohen et al., 2018) developed a blood based assay, CancerSEEK, that assesses levels of circulating proteins and mutations in cell-free DNA to detect eight common cancer types, with sensitivities ranging from 69% to 98%. Biomarkers to diagnose pan-cancer tumours are yet to be identified, however their eventual discovery could offer huge advantages for early detection and optimal clinical follow-up.

Our lab has a long history with the Gasdermin E (GSDME) gene, which was originally identified as being implicated in an autosomal dominant form of hearing loss and named Deafness Autosomal Dominant 5 (DFNA5) (Van Laer et al., 1998). More recently, its function as a tumour suppressor, through the activation of programmed cell death, was revealed (Rogers et al., 2017). The epigenetics of GSDME have been studied in several contexts; some studies have examined its epigenetic silencing through methylation in gastric and colorectal tumours (Akino et al., 2006; Kim et al., 2008; Yokomizo et al., 2012), while more recent studies by our laboratory have highlighted it as a potential methylation-based biomarker for breast cancer (Croes et al., 2017, 2018). Lately, interest in this gene has been rekindled by studies exploring the mechanisms by which it induces cell death, again highlighting its important role to cancer formation. Based on the exceptional in-silico performance of GSDME methylation as a diagnostic/early detection marker in breast and colorectal cancers, we postulated that its methylation patterns could be ubiquitous across several cancer types, a characteristic that could be leveraged for use as a “pan-cancer” biomarker. We further hypothesize that GSDME may likely possess distinctive methylation patterns in the different tumours. Our study aimed to analyze GSDME methylation patterns in the largest cancer patient dataset to date (N=6502) using publicly available data from The Cancer Genome Atlas (TCGA). We thus aimed to assess the capacity of GSDME methylation patterns to serve as effective detection biomarkers in both a pan-cancer and tumour-specific context. In particular, the inventors have now found that by evaluating the methylation status of at least 2 CpG sites in the GSDME gene in a DNA sample from a biological sample, a differential diagnosis between several cancer types is possible.

### SUMMARY

The inventors of the present application have found that the methylation status of the GSDME gene functions as a biomarker for the differential diagnosis between several cancer types, as further also corroborated by the experimental section. In a specific aspect, the inventors identified that the methylation status of at least 2 CpG sites in the GSDME gene; in particular at least 2 CpG site selected from Table 1 in the GSDME gene functions as a biomarker for the differential diagnosis between several cancer types.

TABLE 1
Table showing a simplified reference to the Illumina
Infinium HumanMethylation450 probes, along with
their genomic locations (Genome build h19/GRCh37).

| Probe | Probe | Genomic | |
|---|---|---|---|
| Abbreviation | Name | Coordinate | Location |
| CpG1 | CpG17790129 | 24738572 | Gene body |
| CpG2 | CpG14205998 | 24748668 | Gene body |
| CpG3 | CpG04317854 | 24762562 | Gene body |
| CpG4 | CpG12922093 | 24767644 | Gene body |
| CpG5 | CpG17569154 | 24781545 | Gene body |
| CpG6 | CpG19260663 | 24791121 | Gene body |
| CpG7 | CpG09333471 | 24796355 | Putative |
| | | | Promoter |
| CpG8 | CpG00473134 | 24796494 | Putative |
| | | | Promoter |
| CpG9 | CpG03995857 | 24796553 | Putative |
| | | | Promoter |
| CpG10 | CpG07320646 | 24796981 | Putative |
| | | | Promoter |
| CpG11 | CpG07293520 | 24797192 | Putative |
| | | | Promoter |
| CpG12 | CpG04770504 | 24797363 | Putative |
| | | | Promoter |
| CpG13 | CpG24805239 | 24797486 | Putative |
| | | | Promoter |
| CpG14 | CpG01733570 | 24797656 | Putative |
| | | | Promoter |
| CpG15 | CpG25723149 | 24797680 | Putative |
| | | | Promoter |
| CpG16 | CpG22804000 | 24797691 | Putative |
| | | | Promoter |
| CpG17 | CpG07504598 | 24797786 | Putative |
| | | | Promoter |
| CpG18 | CpG15037663 | 24797835 | Putative |
| | | | Promoter |
| CpG19 | CpG19706795 | 24797839 | Putative |
| | | | Promoter |
| CpG20 | CpG20764575 | 24797884 | Putative |
| | | | Promoter |
| CpG21 | CpG06301139 | 24798175 | Upstream |
| | | | Region |
| CpG22 | CpG26712096 | 24798855 | Upstream |
| | | | Region |

Accordingly, in a first aspect, the present application is directed to the use of the methylation status of the GSDME gene as biomarker for the differential diagnosis between several cancer types in a subject. In particular, the present invention relates to a method for the ex vivo differential diagnosis between several cancer types in a subject comprising; a) obtaining a biological sample comprising DNA from said subject; and b) measuring the methylation status of at least 2 CpG sites in the Gasdermin (GSDME) gene in said biological sample, preferably wherein the cancer types are selected from bladder urothelial carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid adenocarcinoma, uterine corpus endometrial carcinoma, and colorectal carcinoma. In a further embodiment, the present invention relates to a method for the ex vivo differential diagnosis between several cancer types in a subject comprising; a) obtaining a biological sample comprising DNA from said subject; and b) measuring the methylation status of at least 3 CpG sites in the GSDME gene in said biological sample. In still a further embodiment, the methylation status of at least 6 CpG sites in the GSMDE gene is determined in the method according to the present invention.

The method according to the different embodiments of the present application allows for the ex vivo differential diagnosis between several cancer types. In a particular aspect of the invention, said method allows for the ex vivo differential diagnosis between bladder urothelial carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid adenocarcinoma, uterine corpus endometrial carcinoma, colorectal carcinoma.

In a further embodiment, the at least 2 CpG sites, the at least 3 CpG sites or the at least 6 CpG sites of which the methylation status is determined in the method according to the invention, are located in the gene body of the GSDME gene, in the putative gene promoter region of the GSDME gene, or in the region upstream of the putative gene promoter region of the GSDME gene.

In still a further embodiment, the method according to the present invention comprises: a) obtaining a biological sample comprising DNA from a subject; and b) measuring the methylation status of at least 3 CpG sites in the GSDME gene in said biological sample, wherein at least 1 CpG site is located in the gene body of the GSDME gene, at least 1 CpG site is located in the putative gene promoter region of the GSDME gene, and at least 1 CpG site is located upstream of the putative gene promoter region of the GSDME gene.

The method according to the present application is further characterized in that a differential methylation status of at least 2 CpG sites in the putative gene promoter region of the GSDME gene is indicative for a differential cancer diagnosis. In another embodiment, the method is characterized in that a differential methylation status of at least 2 CpG sites in the gene body of the GSDME gene or of at least 2 CpG sites in the putative gene promoter region of the GSDME gene is indicative for a differential cancer diagnosis.

In still a further embodiment, in the methods according to the present invention the CpG sites are selected from the CpG sites listed in Table 1.

In a further aspect, a method for the ex vivo differential diagnosis between several cancer types in a subject is provided, said method comprising: a) obtaining a biological sample comprising DNA from said subject; and b) measuring the methylation status of at least 6 CpG sites in the GSDME gene in said biological sample, wherein the cancer types are selected from bladder urothelial carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid adenocarcinoma, uterine corpus endometrial carcinoma, colorectal carcinoma, and wherein said at least 6 CpG sites are selected from CpG 3, CpG 11, CpG12, CpG13, CpG14, CpG 18, CpG19, CpG20, and CpG21 of Table 1; preferably selected from CpG 3, CpG12, CpG14, CpG18, CpG20, and CpG21 of Table 1.

In another aspect, a method for the ex vivo differential diagnosis between several cancer types in a subject is provided, said method comprising: a) obtaining a biological sample comprising DNA from said subject; and b) measuring the methylation status of at least 6 CpG sites in the GSDME gene in said biological sample, wherein the cancer types are selected from bladder urothelial carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid adenocarcinoma, uterine corpus endometrial carcinoma, colorectal carcinoma, and:
wherein methylation at sites CpG3, CpG12, CpG14, CpG18, CpG20 and CpG21 of Table 1 is indicative for bladder urothelial cancer in the subject, and/or
wherein methylation at sites CpG 2, CpG 3, CpG 4, CpG 14, CpG 17 and CpG 20 of Table 1 is indicative for breast cancer in the subject; and/or
wherein methylation at sites CpG 3, CpG 6, CpG 9, CpG 18, CpG 20 and CpG 22 of Table 1 is indicative for colorectal cancer in the subject; and/or
wherein methylation at sites CpG 1, CpG 3, CpG 7, CpG 11, CpG 14 and CpG 15 of Table 1 is indicative for esophageal cancer in the subject; and/or
wherein methylation at sites CpG 4, CpG 6, CpG 7, CpG 16, CpG 19 and CpG 20 of Table 1 is indicative for head and neck squamous cell carcinoma in the subject; and/or
wherein methylation at sites CpG 3, CpG 7, CpG 15, CpG 19, CpG 21 and CpG 22 of Table 1 is indicative for kidney renal clear cell carcinoma in the subject; and/or
wherein methylation at sites CpG 4, CpG 7, CpG 10, CpG 14, CpG 18 and CpG 22 of Table 1 is indicative for kidney renal papillary carcinoma in the subject; and/or
wherein methylation at sites CpG 3, CpG 5, CpG 6, CpG 7, CpG 13 and CpG 19 of Table 1 is indicative for liver hepatocellular carcinoma in the subject; and/or
wherein methylation at sites CpG 4, CpG 5, CpG 13, CpG 16, CpG 18 and CpG 21 of Table 1 is indicative for lung adenocarcinoma in the subject; and/or
wherein methylation at sites CpG 5, CpG 7, CpG 14, CpG 16, CpG 19 and CpG 20 of Table 1 is indicative for lung squamous cell carcinoma in the subject; and/or
wherein methylation at sites CpG 1, CpG 2, CpG 7, CpG 13, CpG 15 and CpG 22 of Table 1 is indicative for pancreatic adenocarcinoma in the subject; and/or
wherein methylation at sites CpG 1, CpG 3, CpG 10, CpG 14, CpG 16 and CpG 22 of Table 1 is indicative for prostate adenocarcinoma; and/or
wherein methylation at sites CpG 5, CpG 6, CpG 8, CpG 11, CpG 13 and CpG 21 of Table 1 is indicative for thyroid carcinoma; and/or
wherein methylation at sites CpG 1, CpG 5, CpG 14, CpG 15, CpG 16 and CpG 18 of Table 1 is indicative for uterine corpus endometrial carcinoma.

The method according to the different embodiments of the application allows for the ex vivo differential diagnosis between several cancer types. In a further embodiment of the invention, the methylation status of the at least 2 CpG sites, the at least 3 CpG sites or the at least 6 CpG sites in the GSDME gene of the subject is compared to a reference value. In particular, in said method, an altered level of methylation status for said subject relative to said reference value provides an indication that the subject has cancer. In yet another embodiment, in said method, an altered level of methylation for said subject relative to said reference value provides an indication about the cancer type in said subject.

Accordingly, in a further aspect, the present invention is directed to the use of the methylation status of at least 6 CpG sites in the GSDME gene for the ex vivo diagnosis of cancer in a subject. In particular, the invention relates to a method for the ex vivo diagnosis of cancer in a subject, said method comprising: a) obtaining a biological sample comprising DNA from said subject, and b) measuring the methylation status of at least 2; preferably at least 6 CpG sites in the GSDME gene in said biological sample, wherein said CpG sites are selected from the CpG sites listed in Table 1.

In a further aspect, the present invention is directed to a method for the ex vivo diagnosis of cancer in a subject, said method comprising: a) obtaining a biological sample comprising DNA from said subject, and b) measuring the methylation status of 6 CpG sites in the GSDME gene, wherein said CpG sites are selected from the CpG sites listed in Table 1.

In a further embodiment, said at least 6 CpG sites or said 6 CpG sites in the GSDME gene that are selected from Table 1 are CpG 3, CpG 12, CpG 14, CpG 18, CpG 20 and CpG 21 of Table 1.

In another embodiment said at least 6 CpG sites or said 6 CpG sites in the GSDME gene that are selected from Table 1 are CpG 3, CpG 12, CpG 14, CpG 18, CpG 20, CpG 21, CpG 11, CpG 13, and CpG 19 of Table 1.

In another further aspect of the present application, a method for the ex vivo diagnosis of bladder urothelial cancer in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 3, CpG 5, CpG 6, CpG 7, CpG 19, and CpG 22 of Table 1. In a further embodiment, a method for the ex vivo diagnosis of bladder urothelial cancer in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 3, CpG 5, CpG 6, CpG 7, CpG 19, and CpG 22 of Table 1.

In another aspect of the present application, a method for the ex vivo diagnosis of breast cancer in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 2, CpG 3, CpG 4, CpG 14, CpG 17, and CpG 20 of Table 1. In a further embodiment, a method for the ex vivo diagnosis of breast cancer in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 2, CpG 3, CpG 4, CpG 14, CpG 17, and CpG 20 of Table 1.

In another further aspect of the present application, a method for the ex vivo diagnosis of colorectal cancer in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 3, CpG 6, CpG 9, CpG 18, CpG 20, and CpG 22 of Table 1. In a further embodiment, a method for the ex vivo diagnosis of colorectal cancer in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 3, CpG 6, CpG 9, CpG 18, CpG 20, and CpG 22 of Table 1.

In still a further aspect of the present application, a method for the ex vivo diagnosis of esophageal cancer in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 1, CpG 3, CpG 7, CpG 11, CpG 14 and CpG 15 of Table 1. In a further embodiment, a method for the ex vivo diagnosis of esophageal cancer in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 1, CpG 3, CpG 7, CpG 11, CpG 14 and CpG 15 of Table 1.

In still another aspect of the present application, a method for the ex vivo diagnosis of head and neck squamous cell carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 4, CpG 6, CpG 7, CpG 16, CpG 19 and CpG 20 of Table 1. In a further embodiment, a method for the ex vivo diagnosis of head and neck squamous cell carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 4, CpG 6, CpG 7, CpG 16, CpG 19 and CpG 20 of Table 1.

In still another aspect of the present application, a method for the ex vivo diagnosis of kidney renal clear cell carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 3, CpG 7, CpG 15, CpG 19, CpG 21 and CpG 22 of Table 1. In a further embodiment, a method for the ex vivo diagnosis of kidney renal clear cell carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 3, CpG 7, CpG 15, CpG 19, CpG 21 and CpG 22 of Table 1.

In still another aspect of the present application, a method for the ex vivo diagnosis of kidney renal papillary carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 4, CpG 7, CpG 10, CpG 14, CpG 18 and CpG 22 of Table 1. In a further embodiment, a method for the ex vivo diagnosis of kidney renal papillary carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 4, CpG 7, CpG 10, CpG 14, CpG 18 and CpG 22 of Table 1.

In still another aspect of the present application, a method for the ex vivo diagnosis of liver hepatocellular carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 3, CpG 5, CpG 6, CpG 7, CpG 13 and CpG 19 of Table 1. In a further embodiment, a method for the ex vivo diagnosis of liver hepatocellular carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 3, CpG 5, CpG 6, CpG 7, CpG 13 and CpG 19 of Table 1.

In still another aspect of the present application, a method for the ex vivo diagnosis of lung adenocarcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 4, CpG 5, CpG 13, CpG 16, CpG 18 and CpG 21 of Table 1. In a further embodiment, a method for the ex vivo diagnosis of lung adenocarcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 4, CpG 5, CpG 13, CpG 16, CpG 18 and CpG 21 of Table 1.

In still another aspect of the present application, a method for the ex vivo diagnosis of lung squamous cell carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 5, CpG 7, CpG 14, CpG 16, CpG 19 and CpG 20 of Table 1. In a further embodiment, a method for the ex vivo diagnosis of lung squamous cell carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 5, CpG 7, CpG 14, CpG 16, CpG 19 and CpG 20 of Table 1.

In still another aspect of the present application, a method for the ex vivo diagnosis of pancreatic adenocarcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 1, CpG 2, CpG 7, CpG 13, CpG 15 and CpG 22 of Table 1. In a further embodiment, a method for the ex vivo diagnosis of pancreatic adenocarcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 1, CpG 2, CpG 7, CpG 13, CpG 15 and CpG 22 of Table 1.

In still another aspect of the present application, a method for the ex vivo diagnosis of prostate adenocarcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 1, CpG 3, CpG 10, CpG 14, CpG 16 and CpG 22 of Table 1. In a further embodiment, a method for the ex vivo diagnosis of prostate adenocarcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 1, CpG 3, CpG 10, CpG 14, CpG 16 and CpG 22 of Table 1.

In another aspect of the present application, a method for the ex vivo diagnosis of thyroid carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 5, CpG 6, CpG 8, CpG 11, CpG 13 and CpG 21 of Table 1. In a further embodiment, a method for the ex vivo diagnosis of thyroid carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 5, CpG 6, CpG 8, CpG 11, CpG 13 and CpG 21 of Table 1.

In another aspect of the present application, a method for the ex vivo diagnosis of uterine corpus endometrial carcinoma in a subject is disclosed. Said method comprises: a) obtaining a biological sample comprising DNA from said subject, and b) measuring the methylation status of at least 6 CpG sites in the GSDME gene in said biological sample, wherein said at least 6 CpG sites are selected from CpG 1, CpG 5, CpG 14, CpG 15, CpG 16 and CpG 18 of Table 1. In a further embodiment, a method for the ex vivo diagnosis of uterine corpus endometrial carcinoma in a subject is disclosed wherein said method comprises a) obtaining a biological sample comprising DNA from said subject, and b) measuring the methylation status of 6 CpG sites in the GSDME gene in said biological sample, wherein said 6 CpG sites are CpG 1, CpG 5, CpG 14, CpG 15, CpG 16 and CpG 18 of Table 1.

The methods according to different embodiments of the invention allow for the ex vivo differential diagnosis between several cancer types or ex vivo diagnosis of a specific cancer type. In a further embodiment of said methods, the methylation status of the at least 2; the at least 3; or the at least 6 CpG sites in the GSDME gene of the subject is compared to a reference value. In particular, in said method, an altered level of methylation status for said subject relative to said reference value provides an indication that the subject has cancer. In yet another embodiment, in said method, an altered level of methylation for said subject relative to said reference value provides an indication about the cancer type in said subject.

As already discussed herein above, the methods according to the different embodiments of the invention comprise obtaining a biological sample comprising DNA from a subject and measuring the methylation status of the GSDME gene. Said biological sample can be selected from a tissue sample, a stool sample, a cell sample or a bodily fluid sample. In a further embodiment, said biological sample is a bodily fluid sample that is selected from bile, blood, serum, plasma, urine, saliva, sputum or lung aspirate.

The methods according to the different embodiments of the present invention comprise measuring the methylation status of the GSDME gene in a biological sample comprising DNA. In particular, said DNA is DNA from liquid biopsies, circulating tumor DNA or cell-free DNA; preferably circulating tumor DNA. In another embodiment, said DNA is DNA extracted from tumor tissue.

As also discussed above herein, the methods according to the different embodiments of the invention are for the ex vivo differential diagnosis between several cancer types in a subject or for the ex vivo diagnosis of a specific cancer type, thereby using a biological sample comprising DNA from said subject and based on the methylation status of the GSDME gene. Said subject can be a mammal; preferably said subject is a human subject. In a further embodiment, said subject is an adult human subject.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1. The different datasets used for hypothesis testing and result validation. Methylation and expression (RNAseq and microarray) datasets were obtained from TCGA, whereas additional methylation data was obtained from GEO for biomarker validation. TP: primary tumor, NT: normal tissue, P: paired samples (normal and tumor tissue from same individual), L: left-sided CRC, R: right-sided CRC.

FIG. 2. GSDME methylation differences between representative CpGs in the different sample groups. (A)(B) The two presented CpGs exhibit the most significant differences in methylation levels between normal tissues (N=43) and colorectal adenocarcinomas (N=389). The lines indicate the mean GSDME methylation for each group; for CpG03995857(CpG10), the mean methylation is 0.14 (95% CI: 0.05, 0.23) in the normal tissue and 0.50 (95% CI: 0.03, 0.97) in the tumor tissue, while for CpG12922093(CpG4) these values are at 0.67 (95% CI: 0.31, 1.03) and 0.84 (95% CI: 0.78, 0.91) respectively. (C)(D) CpG25723149(CpG15) is representative of GSDME promoter CpGs, where significant differences in methylation levels between left-sided (N=202) and right-sided (N=187) adenocarcinomas were observed in 15 out of 16 CpGs located in the putative gene promoter. For CpG25723149(CpG15), the mean methylation is 0.57 (95% CI: 0.15, 0.98) in the left colon and 0.70 (95% CI: 0.40, 0.99) in the right colon, while for CpG04317854(CpG3) these values are at 0.78 (95% CI: 0.53, 1.03) and 0.80 (95% CI: 0.60, 1.01) respectively.

FIG. 3. Physical map of the 22 CpGs in GSDME, correlating the chromosomal location with the average methylation values. The upper panel corresponds to the tumor versus normal tissues, while the lower panel corresponds to the different anatomical subgroups (left- and right-sided). Error bars indicate the standard error of the mean. A clear trend can be observed in mean methylation values; normal samples are higher methylated in the gene body as compared to tumor samples while the opposite occurs for CpGs in the promoter region. The last two CpGs, located upstream of the putative gene promoter region, show a methylation pattern similar to intragenic CpGs. In the anatomical subgroups, differential methylation is found only in promoter CpGs, with an increased methylation observed in the right-sided group as opposed to the left-sided.

FIG. 4. Correlation matrix of the methylation a-values in the 22 CpGs of GSDME with genomic features overlay exhibiting a bloc-like distribution. Correlation coefficients are indicated by circle color and size. All correlation coefficients had a p-value greater than 0.05. Two distinct clusters can be seen based on the correlation coefficients of the methylation values; promoter region CpGs form the biggest cluster (14 out of 22) while gene body CpGs for the smaller cluster, CpG21 and CpG22 cluster together and follow closely the pattern of the intragenic CpGs. On average methylation correlation in the putative promoter is stronger than that in the gene body, while the two regions don't correlate as well together. CpGs in the south and north shores comprise a strong enhancer region in the gene, whereas intragenic CpGs are located in region of relatively weak transcription.

FIG. 5. Regression plot for probe methylation as a predictor for gene expression. For each of the four groups, CpG probes with the highest impact on RNAseq expression were first selected through a step-wise linear regression model, these were then used altogether in the final regression model where the slope and p-value were calculated. Thick lines indicate +/− one standard error, thin lines indicate +/− two standard error, while * indicates probes with significant p-values (<0.05). Light shading represents intragenic CpGs, dark shading represents putative promoter CpGs, while the darkest shading represents CpGs upstream of the putative promoter region.

FIG. 6. GSDME CpG methylation as biomarker for colorectal adenocarcinomas. The upper panel shows the ROC curve of the final prediction model taking one CpG in the gene body (CpG4) and one CpG in the gene promoter (CpG17) as predictors and accounting for age. Sensitivity and specificity at various cutoff values for the TCGA dataset are plotted resulting in a 0.95 (95% CI: 0.95, 0.98) AUC. At a set cutoff value of 0.72, sensitivity and specificity were at 93.3% and 93.7% respectively while overall model accuracy was 97.6%. The right panel shows ROC curves for the subsequent validation of the model by three external datasets. The AUCs for the external datasets were very similar to that of the original data thus confirming the diagnostic value of the model and its generalizability over other datasets. The diagonal line represents the line of no discrimination between tumor and normal colorectal tissues.

FIG. 7. Binary logistic regression model performance using 1 CpG predictor versus using 2 CpG predictors in the top 5 most common cancer datasets. Using 2 predictors resulted in better average AUC values overall as more information is supplied to the model for a more accurate prediction.

FIG. 8. Barplot of differential methylation analysis of the 22 GSDME probes. Yes=differential methylation, No=no differential methylation, Hypo=Hypomethylated (as compared to control), Hyper=hypermethylated (as compared to control).

FIG. 9. Countplot showing the number of differentially methylated GSDME probes across the datasets. The right panel corresponds to hypermethylated (DNA methylation beta values of tumour samples are significantly higher than that of normal samples) CpGs while the left panel corresponds to hypomethylated (DNA methylation beta values of tumour are significantly lower than that of normal) CpGs. Please refer to Table 2 for tumour dataset abbreviations.

FIG. 10. Map of the 22 GSDME CpGs showing the average probe methylation and chromosomal location across the different datasets. The size of the dots indicates average methylation while the colour indicates tissue type (NT=normal tissue; TP=normal tissue). Please refer to Table 2 for tumour dataset abbreviations.

FIG. 11. Cleveland plot of the calculated AUCs for 39 probe combinations that satisfy both filters (minimum average AUC=0.84 and minimum AUC threshold=0.80) across the datasets.

FIG. 12. Countplot of the number of tumour types per combination that satisfy the AUC filters.

FIG. 13. Countplot of the number of probe combinations that satisfy the filters for each of the datasets. Please refer to Table 2 for tumour dataset abbreviations.

FIG. 14. ROC curves for the final GSDME pan-cancer model along with the validation datasets. The black solid curve represents the training dataset, the red solid line represents the combined validation dataset, while the dotted lines represent the individual validation sets. The final model included 6 CpG probes; one in the gene body (Probe 3), 4 in the promoter region (Probes 12, 14, 18 and 20) and one in the upstream region (Probe 21) and accounted for age and tumour stage. Sensitivity and specificity at various cut-off values for the datasets are plotted. The final model yielded an AUC of 0.86 (95% CI: 0.852-0.87). At a set cut-off of 0.55, sensitivity and specificity were at 98.8% and 93.2% respectively while overall model accuracy was 89.7%. The right panel show ROC curves for the subsequent validation of the model by 3 external datasets. The diagonal line represents the line of no discrimination between tumour and normal tissues.

FIG. 15. Violin plot of the distribution of PLSDA cross-validated AUCs of different probe combinations (74,613) classifying each of the 14 tumour types against all others. Please refer to Table 2 for tumour dataset abbreviations.

FIG. 16. Flower plot of the maximum calculated cross-validated AUC for classifying each of the 14 tumours against all others, along with the corresponding probe combination that yielded the displayed AUC. Please refer to Table 2 for tumour dataset abbreviations.

FIG. 17. Interaction plot for the RNA-seq expression data showing the differences in expression levels between tumour and normal samples across the different tumour types.

### DETAILED DESCRIPTION

The present invention is based on the finding that differential methylation of the GSDME gene can be used for the ex vivo differential diagnosis between several cancer types in a subject, in particular a human subject.

In particular, the inventors of the present application have found that differential methylation analysis of the GSDME gene can be used to differentiate between 14 different cancer types. Said cancer types include bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, colorectal carcinoma. As evident from the example section below, differential diagnosis between several cancer types is already possible based on the methylation status of at least 2 CpG sites in the GSDME gene; preferably of at least 3 CpG sites in the GSMDE gene; even more preferably of at least 6 CpG sites in the GSDME gene.

Therefore, in a first embodiment, the present invention is directed to a method for the ex vivo differential diagnosis between several cancer types in a subject comprising:
a) obtaining a biological sample comprising DNA from said subject; and
b) measuring the methylation status of at least 2 CpG sites in the GSDME gene in said biological sample,
wherein the cancer types are selected from bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, colorectal carcinoma.

The biological sample may be any sample in which the methylation status of the GSDME gene can be determined. In one aspect, the biological sample is a tissue sample, a stool sample, a cell sample or a bodily fluid sample. In one aspect, the biological sample is a neoplastic tissue sample, such as a tumour sample, e.g. a primary or metastatic tumour sample. The biological sample may also be derived from a biological fluid or body fluid, for example, whole blood, blood, urine, lymph fluid, serum, plasma, nipple aspirate, ductal fluid, saliva, bile, sputum or tumour exudate. It has been shown in the literature that cancer or tumour cells often release genomic DNA in circulating or other bodily fluids. Since said genomic DNA has the same methylation profile as the DNA inside the tumour or cancer cell, said methylation profile can be detected in the circulating or other bodily fluid sample as well. This has for example been reviewed by Qureshi et al., 2010 (Int. J. Surgery 2010, 8:194-198), hereby incorporated by reference in its entirety. In certain embodiments, the biological sample is thus a circulating tumour DNA sample. In other embodiments, the biological sample is a bodily fluid comprising neoplastic cells.

In certain embodiments of the methods of the present invention, the sample is a neoplastic tissue sample. In certain embodiments, the neoplastic tissue sample is a neoplastic tissue biopsy or neoplastic tissue for fine-needle aspirate. In certain embodiments, the neoplastic tissue sample is resected neoplastic tissue. In certain embodiments, the sample is tumour biopsy or tumour fine-needle aspirate, for example biopsy or fine-needle aspirate from primary or metastatic tumour tissue. In other embodiments, the sample is resected tumour tissue, e.g. resected primary or metastatic tumour tissue.

The biological sample can be obtained from a subject in any way typically used in clinical settings for obtaining a sample comprising the required cells or nucleic acid. For example, the sample can be obtained from fresh, frozen, or paraffin-embedded surgical samples or biopsies of an organ or tissue comprising the suiTable cells or nucleic acid to be tested. If desired, the sample can be mixed with a fluid or purified or amplified or otherwise treated. For example, samples may be treated in one or more purification steps in order to increase the purity of the desired cells or nucleic acid in the sample, or they may be examined without any purification steps. Any nucleic acid specimen in purified or non-purified form obtained from such sample can be utilized in the methods according to the present invention.

In certain embodiments, the sample may be a formalin-fixed and paraffin-embedded (FFPE) sample or fresh-frozen sample. Preferably, the sample is a FFPE sample.

The terms “subject”, “individual”, or “patient” are used interchangeably throughout this specification, and typically and preferably denote humans, but also encompass reference to non-human animals, preferably warm-blooded animals, even more preferably mammals, such as e.g. non-human primates, rodents, canines, felines, equines, ovines, porcines, and the like. The term “non-human animals” includes all vertebrates, e.g. mammals, such as non-human primates (particularly higher primates), sheep, dog, rodent (e.g. mouse or rat), guinea pig, goat, pig, cat, rabbits, cows, and non-mammals such as chicken, amphibians, reptiles etc. In certain embodiments, the subject is a non-human mammal. In certain preferred embodiments, the subject is a human subject. In other embodiments, the subject is an experimental animal or animal substitute as a disease model. The term does no denote a particular age or sex. Thus, adult and newborn subjects, as well as foetuses, whether male or female, are intended to be covered.

SuiTable subjects may include without limitation subjects presenting to a physician for a screening for a neoplastic disease, subjects presenting to a physician with symptoms and signs indicative of a neoplastic disease, subjects diagnosed with a neoplastic disease, subjects who have received anti-cancer therapy, subjects undergoing anti-cancer treatment, and subjects having a neoplastic disease that is in remission.

The present invention is directed to a method for the ex vivo differential diagnosis between several cancer types by evaluating the methylation status of at least 2 CpG sites in the GSDME gene in a biological sample from a subject. In a further aspect, said methylation status is compared to a reference value.

In some aspects, said reference value is a baseline level of methylation present in a population of subjects without neoplasia or cancer. In another aspect, said reference value is a baseline level of methylation in the same subject prior to, during or after treatment for a neoplasia or cancer. In another aspect, said reference value is a standardized curve. In still another instance, said reference value represents a range or an index about the methylation status obtained from at least two samples. Said samples can be derived from healthy subjects not afflicted with cancer or pre-forms thereof without neoplasia, or from subjects prior to, during or after treatment for a neoplasia or cancer. The reference value may also represent a neoplastic tissue sample or healthy tissue sample, such as from the same subject or a different subject. Reference values according to all the different embodiments may be established according to known procedures. For example, a reference value may be established in a reference subject or individual or a population of individuals characterized by a particular prediction of cancer risk. Such population may comprise without limitation two or more, 10 or more, 100 or more, or even several hundred or more individuals.

The inventors of the present application also found that the methylation in the GSDME gene occurs in block-like structures. In other words, the methylation pattern of the GSDME gene is organized in clusters situated in three regions of the GSDME gene, namely the gene body of the GSDME gene, the putative gene promoter region of the GSDME gene and the region upstream of the putative gene promoter region of the GSDME gene. Furthermore, the inventors specifically show differential methylation of CpG sites between tumour and normal tissue and between different tumour types, in the gene body of the GSDME gene, in the putative gene promoter region of the GSDME gene and in the region upstream of the putative gene promoter region of the GSDME gene. As is also evidenced in the examples, two distinct clusters of methylation were found to be localized to the gene body and promoter regions.

Based on these findings, in one aspect of the invention, the method for the ex vivo differential diagnosis between several cancer types in a subject comprises obtaining a biological sample comprising DNA from said subject, and measuring the methylation status of at least 2 CpG sites in the gene body of the GSDME gene, of at least 2 CpG sites in the putative gene promoter region of the GSDME gene, and of at least 2 CpG sites located upstream of the putative gene promoter region of the GSDME region. In a preferred embodiment, the methylation status of at least 2 CpG sites in the gene body or in the putative gene promoter region is measured.

In another aspect, detection of a differential methylation status of at least 2 CpG sites in the putative gene promoter region of the GSDME region is indicative for differential cancer diagnosis, in particular for a differential cancer diagnosis for the detection of a specific cancer type selected from bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, and colorectal carcinoma.

In another aspect, detection of a differential methylation status of at least 2 CpG sites in the gene body of the GSDME gene or located upstream of the putative gene promoter of the GSDME region is indicative for differential cancer diagnosis, in particular for a differential cancer diagnosis for the detection of a specific cancer type selected from bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, and colorectal carcinoma.

The present invention also provides an assay or a kit for detecting the methylation status of the GSDME gene. Said assay or kit comprises reagents to perform a methylation- sensitive PCR assay to determine the methylation status of the GSDME gene. Said reagents include primers, buffers, DNA nucleotides and oligonucleotides, restrictions enzymes. Said kit also comprises instructions to perform the method according to any of the different embodiments of the present invention.

In a further aspect, the present invention provides a method for the treatment of a subject susceptible of having cancer. Said method comprises the differential diagnosis between several cancer types selected from bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, and colorectal carcinoma, and based on the methylation status of at least 2 CpG sites in the GSDME gene, followed by treatment of the subject with a cancer treatment or a combination of cancer treatments known to be effective for the identified cancer type. In a further aspect, said method of treatment comprises the differential diagnosis between several cancer types based on the methylation status of at least 3; preferably of at least 6 CpG sites in the GSDME gene, followed by treatment of the subject with a cancer treatment or combination of cancer treatments known to be effective for the identified cancer type.

The present is invention is now further disclosed in the following examples:

### EXAMPLES

### Example 1: Methylation of GSDME Gene in Colorectal Cancer

### Materials and Methods

Datasets and Study Population

The analyses presented in this example were carried out on TCGA (colon and rectum adenocarcinoma) datasets that were downloaded from the GDC data portal website (portal.gdc.cancer.gov) using an in-house developed Python script. The script merely automates the querying of TCGA in order to easily and quickly download the data. TCGA stores patient sample data under unique barcodes following a specific layout; these are used to access biological and clinical data in the database. First, all patient barcodes available for colorectal cancer were downloaded via the website. API URLs were generated using the downloaded barcodes in order to query the matching TCGA level 3 methylation 450k Illumina platform data, the RNAseq V2 gene expression data and the Agilent 244K microarray expression data. Subsequently, the methylation and gene expression data were downloaded for each barcode (patient) and stored in separated JSON formatted files. The individual JSON files were then merged per data type (methylation, RNAseq expression and microarray expression), through Python's dictionary functionality. This resulted in three data matrices, where sample data points (values: beta-value or normalized counts) were column-wise concatenated using the row name features (keys: methylation probe names or official gene symbols, 450K methylation or RNAseq V2 data respectively). The end result is a large Table with probes row-wise and samples column-wise. The same principle was applied for downloading biospecimen and clinical data files. The biospecimen in the TCGA datasets were flash frozen/formalin-fixed paraffin-embedded, resection tissue samples, containing a minimum of 60% tumor nuclei and derived from primary, untreated colorectal tumor tissue. Using the in-house script, methylation (level 3) data was obtained from the portal for all 22 GSDME CpGs.

Six of these CpGs (CpG1-CpG6) are located in the gene body which extends from exon 2 until exon 10, 14 (CpG7-CpG20) are located in the putative gene promoter which lies upstream of exon 2, while the last two (CpG21-CpG22) are located in the upstream region, the details of which are described in Table 1. Methylation is reported as β-value, which is the ratio of the methylated probe intensity over the sum of methylated and unmethylated probe intensities, ranging from zero to one. These values were obtained by TCGA using the Illumina Infinium HumanMethylation450 BeadChip microarrays (Illumina Inc., San Diego, Calif., USA). RNA sequencing (RNAseq) and microarray expression datasets were obtained in a similar fashion. RNAseq expression values in TCGA were acquired using the IlluminaHiSeq platform (Illumina, San Diego, Calif., USA), and the respective transcript abundances were quantified using the Expectation Maximization algorithm. The expression values are reported as log2 transformed value and the highest predicted transcript for GSDME in RNAseq was the most abundant (NM_004403), while the expression of the other transcripts was negligible. Microarray expression values were obtained in TCGA using the Agilent 244K Custom Gene Expression G4502A-07® microarrays (Agilent, Santa Clara, Calif., USA) that contain two probes for GSDME (A_23_P82448 [36.3:chr.7:24705001-24705060] and A_23_P82449 [36.3:chr.7:24705092-24705151]), covering the three most abundant GSDME transcripts (NM_004403, NM_001127454.1, NM_001127453.1). Transcript NM_004403.2 was the most abundant, while the expression of the other transcripts was negligible and hence could not be included in the study. All microarray expression values are expressed as log2 transformed fold changes relative to the Universal Human Reference RNA (Stratagene).

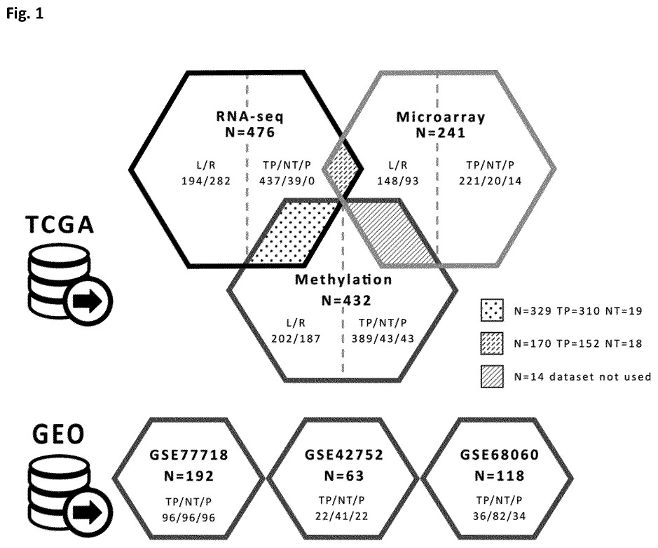

Primary tumor samples for which clinical data was available were then split into two categories; “left-sided” and “right-sided”, based on the anatomical location of the neoplasm, with the splenic flexure acting as the demarcation line between the two categories. Inherently, samples taken from the caecum, ascending colon, hepatic flexure and transverse colon were part of the right-sided category, while samples from the splenic flexure, descending colon, sigmoid colon, rectosigmoid junction and rectum comprised the left-sided category. This categorization is based on the pragmatic split between the embryological origins of the colorectal tissue such that the right part of the colon originates from the midgut, while the left part is derived from the hindgut. After data filtering and classification, several final datasets were available for the downstream analyses. Information about these datasets is found in FIG. 1 (FIG. 1 prio1).

Statistical Analyses

We designated the following clinicopathological parameters from the TCGA clinical patient data files with which to carry out association analyses: age at diagnosis, gender, pathological tumor stage (I-IV), anatomic neoplasm subdivision (left-sided or right-sided), and presence of colon polyps at procurement (Table 1). The GSDME sequence regions, methylation probe locations and chromatin states were explored using the UCSC genome browser. The statistical software R (version 3.4.1) was used to carry out all the statistical analyses. All reported p-values are two-sided, and those less than or equal to 0.05 were considered statistically significant. To account for the non-independence between measurements from the same individuals, a linear mixed model was fitted and included a random effect for sample barcodes. The significance of the fixed effects was then tested via the F-test with a Kenward-Roger correction for the number of degrees of freedom. Differences between groups were assessed through t-tests and linear regression models, while association between expression and CpG methylation was tested using Spearman's correlation and through linear regression models. In all regression models age was accounted for as a covariate, but it was excluded from the final model if its effect on the outcome was not significant.

Five-year overall survival analysis was carried out by fitting Cox proportional hazard models to the methylation and expression datasets and including age as covariate. Additionally, stratified Cox models with separate baseline hazards for the four tumor stages were fitted. Censoring was carried out for individuals who died after the five year (1826 days) mark of the analysis and their respective follow up time was set to 1826 days. Quantile-quantile plots showing the distribution of the 22 observed p-values as compared to the uniform distribution, which is expected in the absence of any true association signal, were generated.

To assess the viability of GSDME methylation and expression as a biomarker for colorectal cancer, binary logistic regression was fitted to predict tissue type (normal/tumor) based on methylation and expression values with age as covariate. Stepwise multiple regression analysis was carried out to determine the best combination of the 22 CpGs. The final model was chosen based on the best Akaike information criterion (AIC) values with the lowest number of predictors possible. The accuracy of the model predictions was assessed by plotting receiver operating characteristic (ROC) curves. A ten-fold cross validation of these results was then performed. Moreover, three additional Illumina 450K CpG methylation datasets were downloaded from the Gene Expression Omnibus (GEO) database (www.ncbi.nlm.nih.gov/geo/) (GEO accession numbers GSE77718, GSE42752, and GSE68060), and were used for the subsequent external validation (FIG. 1). The final model was refit on each of the external datasets and the AUC was recalculated for the new predictions. The same methodology was also applied to RNAseq and microarray datasets to determine their predictive potential.

### Results

GSDME Methylation and Expression in Primary Untreated Colorectal Adenocarcinomas and Histologically Normal Colorectal Tissue

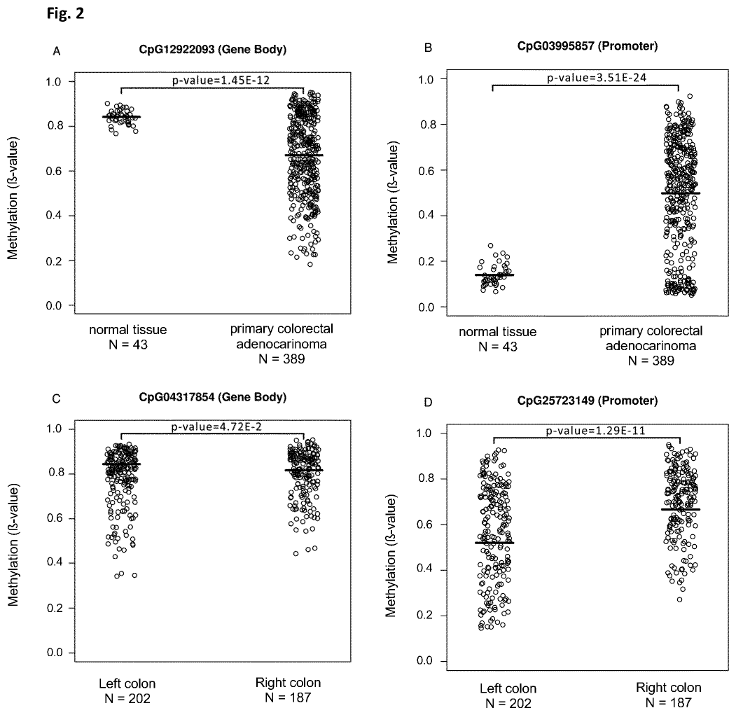

Our results showed a significant methylation difference between primary tumor and normal colorectal tissue for all 22 CpGs in the non-paired samples (p-value=3.51E-24 to 3.94E-2) and in 19 of 22 CpGs in the paired samples (p-value=1.65E-16 to 2.53E-2) (data not shown). For the significantly different CpGs located in the gene body, methylation levels in the normal tissue were higher than those in the tumor tissue, while the opposite holds true for CpGs located in the putative promoter region. The pattern switched again with two CpGs (CpG21 and CpG22), located upstream of the putative gene promoter region; these again showed increased methylation in the normal tissue as opposed to the tumor tissue (FIGS. 2A-B).

Two sources of GSDME expression were examined: RNAseq and microarray. The mean RNAseq expression for the normal tissues (5.80 95% CI: 3.31, 8.29) was slightly higher than that for the tumor tissues (5.45 95% CI: 2.68, 8.22), but these differences were not significant neither for the paired nor for the un-paired samples. The same held true for microarray data where no significant differences were observed between the normal and tumor tissues (means of −3.18, 95% CI: −5.89, −1.38 and −0.46, 95% CI: −4.79, −1.38 respectively). Additionally, we explored the correlation between the two sources of GSDME expression data in samples for which both microarray and RNAseq GSDME expression values were available. The two datasets were highly correlated with a Spearman's coefficient of 0.89 for the whole datasets, 0.85 and 0.84 for the tumor tissues and normal tissues respectively, and 0.88 and 0.86 for the left-sided and right sided groups respectively, all of which having a p-value<2.2e-16. With respect to the mentioned clinicopathological parameters, only age had a significant effect on the methylation of 14 out of 22 probes. These were CpGs 7-18, 20 and 22. The calculated regression slopes were very close to zero (0.0002-0.005) and as such the positive effect of age on probe methylation was somewhat minor. These same CpGS showed significant, although weak (0.1-0.2), positive correlation coefficients, with the exception of CpG22 that had a weak negative correlation coefficient (−0.1).

GSDME Methylation and Expression in Left-Sided and Right-Sided Colorectal Adenocarcinomas

With respect to left-sided and right-sided colorectal adenocarcinomas, our investigation showed a significant difference in methylation levels between the subgroups for 18 out of 22 CpGs (p-value=1.66E-13 to 4.71E-2). Interestingly, most significant differences were observed in the putative promoter region (CpG6-22), whereas only two CpGs in the gene body were significantly different in methylation between the two groups (CpG1, p-value=4.21E-2 and CpG3, p-value 4.71E-2). For the significant CpGs, the methylation levels in the left-sided subgroup were consistently lower than those in the right-sided group and followed the general trend of putative promoter CpGs in the normal colorectal tissue (FIG. 2C-D). No significant differences in GSDME expression between the two groups were found. The correlation between methylation and expression in the left-sided subgroup was 0.86 while in the right-sided subgroup it was 0.84.

GSDME Methylation and Genomic Location

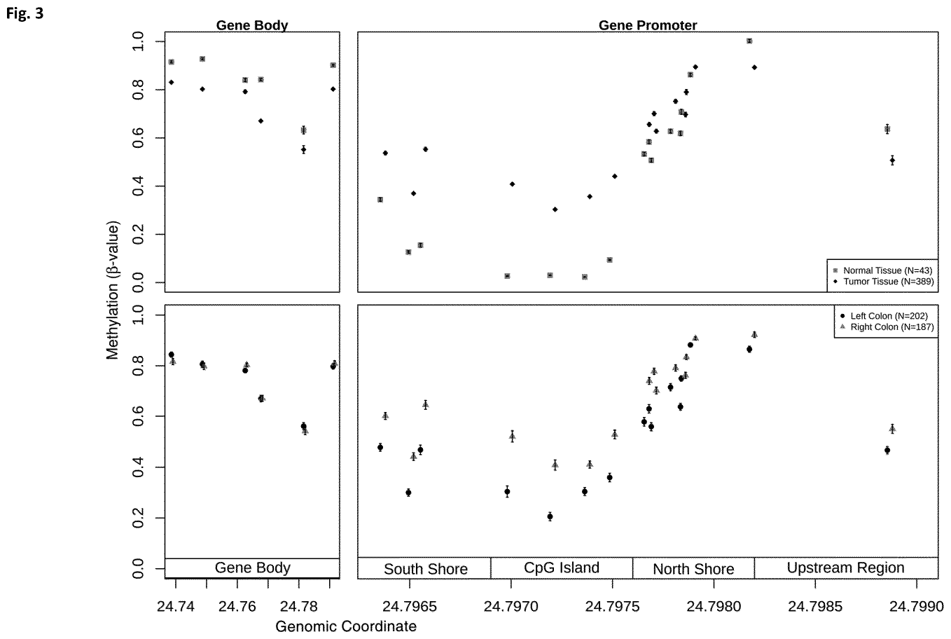

After plotting the average GSDME methylation per CpG versus the respective physical map position on chromosome seven (human genome build 37), a clear trend in methylation was further elucidated (FIG. 3). Methylation levels of the first six CpGs, located in the gene body, are higher in normal colorectal tissue as compared to tumor tissue. Conversely, the 14 following CpGs, located in the putative promoter region displayed a consistently lower methylation in the normal tissue as compared to tumor tissue. The inverse of this methylation pattern was seen for the last two CpGs, which are located upstream of the putative gene promoter region. As for the left-sided and right-sided groups, no difference can be seen in the methylation of gene body CpGs, in the putative promoter region the left-sided group shows lower methylation compared to its counterpart (FIG. 3).

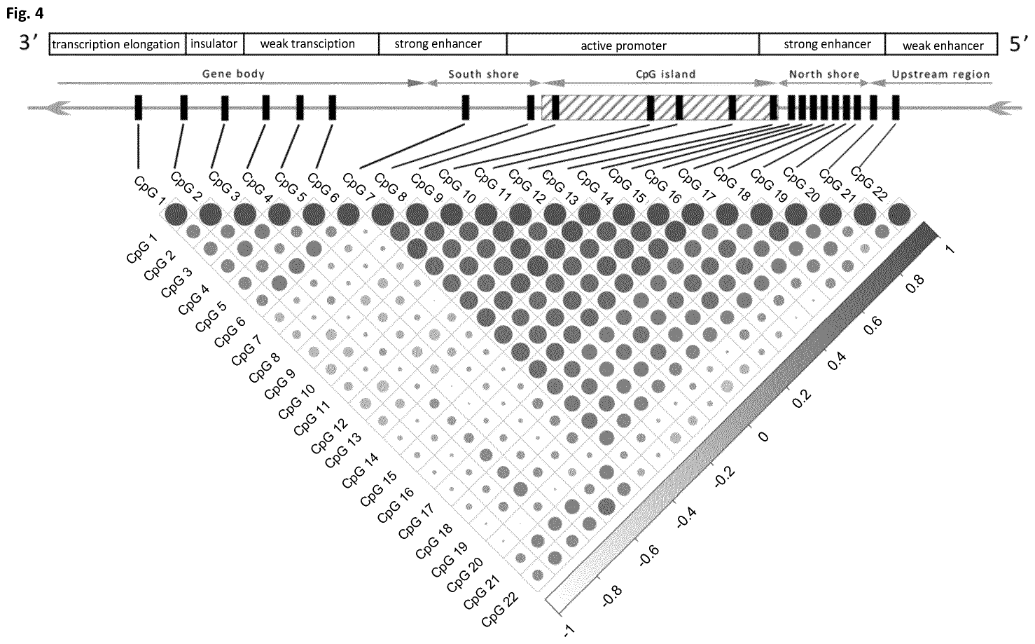

A correlation matrix for the methylation values of all 22 CpGs to investigate the association between the methylation of different regions in the GSDME gene, showed a block-like clustering; a smaller cluster made up of the six CpGs located in the gene body, and a larger cluster made up of the remaining 14 CpGs located in the putative gene promoter region (FIG. 4). Additionally, the last two CpGs located upstream of the putative gene promoter region clustered together and had a pattern similar to the gene body cluster. In these clusters, the larger CpG group, pertaining to probes in the putative promoter region, had the largest positive pairwise correlation coefficients whereas the smaller group had lower positive coefficients, all of which having significant p-values less than 0.05 (FIG. 4).

Moreover, an accumulation of methylation was observed in the promoter region of tumor tissues with a significant 32% increase over the normal tissues. When excluding CpGs 21 and 22, which are thought to be upstream of the putative promoter region and clearly follow the methylation patterns of gene body CpGs (FIG. 4), a 43% increase in methylation is observed. With respect to the gene body, a 13% decrease in methylation is observed in the tumor tissues as opposed to the normal. CpG islands are normally larger than 200 bp in length with a GC content above 50%. Shore regions are located up to two kilo base pairs upstream or downstream from the CpG island, while shelfs are regions two to four kilo base pairs away from the island. Based on the UCSC genome browser, a 946 bp CpG island was found to be part of the putative promoter, flanked by two enhancer regions (FIGS. 3 and 4). Moreover, high DNAse I activity is reported around the putative promoter region along with binding sites for E2F1 and Po1R2A transcription factors.

Based on this delimitation and on the strong correlation in methylation patterns between CpGs located in the same genomic regions (FIG. 4), the two distinct clusters of methylation can be localized to the gene body and promoter regions. The former includes the first 6 CpGs while the latter includes the following 14. The 2 remaining CpGs, that are in a region upstream of the promoter, closely follow the methylation pattern of gene body CpGs and hence belong to that bloc.

Association Between GSDME Methylation and Expression

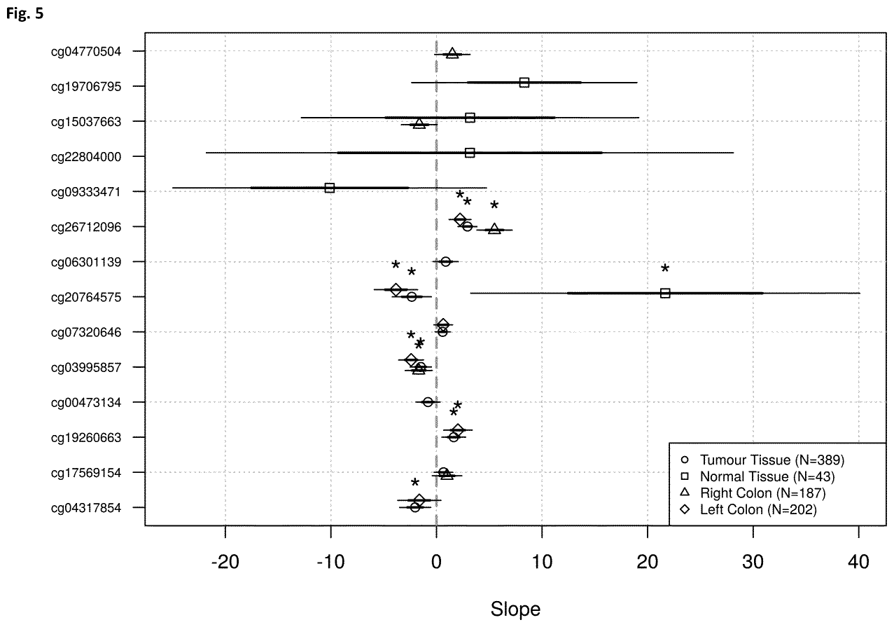

We calculated the Spearman correlation coefficient to study the association between GSDME methylation and expression in samples for which both methylation data and expression data was available (RNAseq dataset), but none of the calculated correlation coefficients were strong. Regression analysis over the whole dataset resulted in significant p-values for CpGs 3, 6, 9, 20 and 22, this association however, was very weak indicated by the small exploratory variable slopes (FIG. 5). Regression analysis on the grouped samples, showed that for the tumor samples about 40% of the variance in GSDME expression was attribuTable to variance in GSDME methylation (R²=0.38, model p-value=2.20E-16). Five CpGs (CpG3, CpG6, CpG9, CpG20, CpG22) showed significant association between methylation p-value and RNAseq expression. In the normal samples, a regression model could be fit, explaining around 60% of the variance in expression (R²=0.63, model p-value=1.10E-2); however, only one CpG (CpG20 p-value=3.50E-2) showed a significant association with GSDME expression. In the anatomical subgroups, around 40% of the variance could also be explained by the CpGs included in the models. In the left-sided group the methylation of only two CpGs (CpG9, CpG22) showed a significant association with GSDME expression, while in the right-sided group four CpGs (CpG6, CpG9, CpG20, CpG22) were significantly associated. Overall, the regression analysis showed a heterogeneity in the effects of CpG methylation on expression. The coefficients were spread between positive and negative values with most of them clustering around zero, indicating minor effects between the variables (FIG. 5). The results in both datasets are relatively disparate and hence the contribution of GSDME methylation to expression levels is still inconclusive, with no consistent association between the two.

Associations Between GSDME Methylation or Expression and 5-Year Overall Survival

The association between survival and methylation or expression was studied using Cox proportional-hazard models in patients for which follow-up data was available (N=260). For the complete adenocarcinoma dataset, no significant association between methylation and 5-year survival could be found. For the left-sided and right-sided subgroups, a significant association was found only for one CpG (CpG22 p-value=1.60E-02) and for two CpGs (CpG4 p-value=1.51E-2, CpG21 p-value=3.13E-2) respectively. By comparing the distribution of the p-values to the expected distribution under the null hypothesis of no association, no enrichment in low p-values was observed and hence CpG methylation does not seem to be a strong predictor of 5-year survival. We repeated the same analysis for both RNAseq and microarray expression data, but again no clear association could be deduced. It is noteworthy that in all hazard proportion models, only age had a significant influence on survival.

GSDME Methylation and Expression as Potential Detection Biomarker for Colorectal Adenocarcinomas

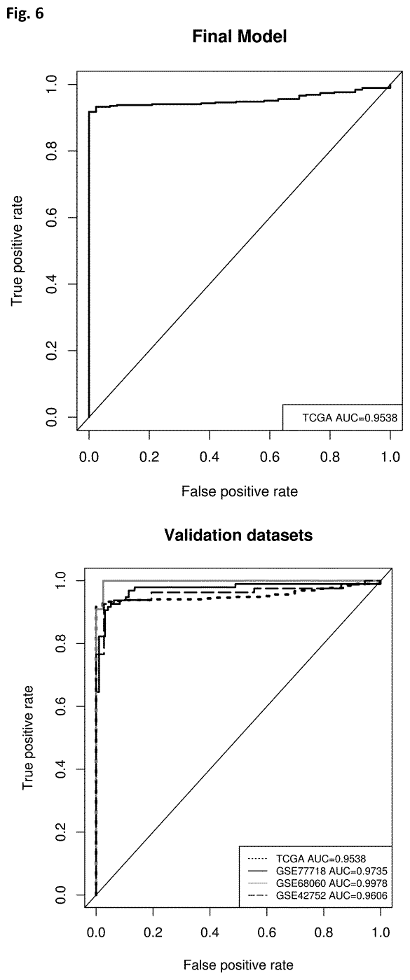

In a logistic regression framework, we explored all combinations of the 22 CpGs that would yield discriminatory power to distinguish between tumor and non-tumor tissue states. Six CpGs had good predictive value in our models (CpG12, CpG14, CpG4, CpG17, CpG15, CpG2). In general, models with two CpGs led to a better prediction than those with only one. Their AUC values were in the range of 0.72-0.97 and 0.71-0.87 respectively (FIG. 6, Table 1). To analyze if the relation between CpG methylation and disease status (or tissue type) is homogeneous across tumor stages, we fitted logistic regression models. Tissue type was entered as dependent variable, and independent variables included CpG methylation, stage and the interaction between methylation and stage. The significance of this latter term tests the null hypothesis of homogeneity of the marker across the stages: in case the p-value of the interaction is significant, the association between the CpG methylation and the tissue type is not uniform across stages. The significance of the interaction term was tested using a likelihood ratio test, comparing the fit of the model with both main effects and their interaction term, against the model with only the main effects of methylation and stage. None of the stages or interaction terms showed a significant outcome on tissue type prediction.

For our final prediction model, CpG 12 located in the putative promoter region and CpG4 located in the gene body were chosen as predictors, resulting in a 0.95 (95% CI: 0.95-0.98) AUC value. A 10-fold cross-validation showed an AUC value of 0.95 (95% CI: 0.93-0.97, StdErr=0.01). Sensitivities and specificities at the different cutoff values for the predicted probabilities are shown by means of an ROC plot (FIG. 6). At a cutoff value of 0.72, a sensitivity of 93.3% and a specificity of 93.7% for detection of colorectal adenocarcinomas were reached without false positives, with an overall accuracy of 97.6%. As an external validation, we applied our trained model to three external CpG methylation datasets downloaded from the GEO database (FIG. 1) using the same two CpGs as predictors. Sample tissue type was successfully predicted in all of the three datasets with AUC values comparable to that of the original TCGA dataset; GSE77718, GSE42752, and GSE68060 had AUCs of 0.97, 0.96 and 0.99 respectively. In all, the model exhibited a high predictive power and good generalisability across different datasets (FIG. 6, Table 1).

We additionally investigated the potential of GSDME expression data as a biomarker. Using the same methodology, a ROC curve was constructed using RNAseq data for 453 tumor tissues and 41 normal tissues and microarray data for 221 tumor tissues and 20 normal tissues. The AUC values were 0.55 and 0.60 respectively, reflecting a low predictive power.

### Example 2: GSDME Methylation as a Pan-Cancer and Cancer-Type Specific Biomarker

### Materials and Methods

Datasets and Study Population

The analyses presented here were carried out on TCGA datasets that were downloaded from the GDC data portal website (portal.gdc.cancer.gov) using an in-house developed Python script. First, all patient barcodes available were downloaded via the website. Level 3, 450K DNA methylation data and RNAseq V2 gene expression data, were downloaded from the TCGA Data Portal (tcga-data.nci.nih.gov) using an in-house developed Python (version 2.7) script as described in (Ibrahim et al., 2019). Methylation data was downloaded for each barcode (patient) and stored in separated JSON formatted filed. The individual JSON files were then merged per data through Python's dictionary functionality. This resulted in three data matrices, where sample data points (values: beta-values) were column-wise concatenated using the row name features (keys: methylation probe names). The end result is a large Table with probes row-wise and samples column-wise. Biospecimen in the TCGA datasets were flash frozen/formalin-fixed paraffin-embedded, resection tissue samples, containing a minimum of 60% tumour nucleic and derived from primary, untreated colorectal tumour tissue. Using the described script, methylation (level 3) data was obtained from the portal for all 22 GSDME CpGs for all 33 TCGA study names referring to the different cancer types. Although TCGA houses data for more than 30 different tumours, some of the datasets had too few normal tissues for a valid statistical analysis. We chose datasets that have a minimum case to control ratio of 10% and those have at least 10 control samples. In total, datasets for 15 distinct tumours were downloaded. Colon and rectal tumour datasets were combined to form the colorectal cancer dataset, resulting in 14 unique datasets, the details of which are presented in Table 2. Similarly, biospecimen and clinical data files for the different datasets were also downloaded. Samples in TCGA datasets were flash frozen/formalin-fixed paraffin-embedded, resection tissue samples, containing a minimum of 60% tumour nuclei and derived from primary, untreated tumour tissue.

TABLE 2
Overview of the TCGA datasets used for the analysis.

| Dataset Name (TCGA Abbreviation) | # NT | # TP | # Total |
|---|---|---|---|
| Bladder urothelial | 21 | 418 | 439 |
| carcinoma (BLCA) | | | |
| Breast carcinoma (BRCA) | 96 | 791 | 887 |
| Esophageal carcinoma (ESCA) | 16 | 185 | 201 |
| Head and Neck squamous | 50 | 528 | 578 |
| cell carcinoma (HNSC) | | | |
| Kidney renal clear cell | 160 | 324 | 484 |
| carcinoma (KIRC) | | | |
| Kidney renal papillary | 45 | 275 | 320 |
| cell carcinoma (KIRP) | | | |
| Liver hepatocellular | 50 | 377 | 427 |
| carcinoma (LIHC) | | | |
| Lung adenocarcinoma (LUAD) | 32 | 473 | 505 |
| Lung squamous cell | 42 | 370 | 412 |
| carcinoma (LUSC) | | | |
| Pancreatic adenocarcinoma (PAAD) | 10 | 184 | 194 |
| Prostate adenocarcinoma (PRAD) | 50 | 502 | 552 |
| Thyroid carcinoma (THCA) | 56 | 507 | 563 |
| Uterine Corpus Endometrial | 46 | 438 | 484 |
| Carcinoma (UCEC) | | | |
| Colorectal carcinoma (CRC) | 45 | 411 | 456 |
| Total | 719 | 5783 | 6502 |

NT = control sample;
TP = case sample

Methylation values were obtained by TCGA using the Illumina Infinium HumanMethylation450 BeadChip microarrays (Illumina Inc, San Diego, Calif.). Methylation is reported as β-value, which is the ratio of the methylated probe intensity over the sum of methylated and unmethylated probe intensities, ranging from 0 to 1. The Illumina 450K array includes 22 probes for the GSDME CpG sites, 16 of which are in the putative promoter, four are located in the putative gene body, while the remaining two are located in a region upstream of the putative promoter, the details of which are described in Table 3. A scheme showing the GSDME gene structure and CpG distribution can be found in (Croes et al., 2018; Ibrahim et al., 2019).

TABLE 3
Table outlining the GSDME Illumina Infinium
HumanMethylation450 probes along with their
genomic locations (Genome build h19/GRCh37).

| Probe | Probe Name | Genomic | | |
|---|---|---|---|---|
| Abbreviation | (Illumina) | Coordinate* | Location | Chromosome |
| Probe 1 | CpG17790129 | 24738572 | Gene body | 7 |
| Probe 2 | CpG14205998 | 24748668 | Gene body | 7 |
| Probe 3 | CpG04317854 | 24762562 | Gene body | 7 |
| Probe 4 | CpG12922093 | 24767644 | Gene body | 7 |
| Probe 5 | CpG17569154 | 24781545 | Gene body | 7 |
| Probe 6 | CpG19260663 | 24791121 | Gene body | 7 |
| Probe 7 | CpG09333471 | 24796355 | Putative | 7 |
| | | | Promoter | |
| Probe 8 | CpG00473134 | 24796494 | Putative | 7 |
| | | | Promoter | |
| Probe 9 | CpG03995857 | 24796553 | Putative | 7 |
| | | | Promoter | |
| Probe 10 | CpG07320646 | 24796981 | Putative | 7 |
| | | | Promoter | |
| Probe 11 | CpG07293520 | 24797192 | Putative | 7 |
| | | | Promoter | |
| Probe 12 | CpG04770504 | 24797363 | Putative | 7 |
| | | | Promoter | |
| Probe 13 | CpG24805239 | 24797486 | Putative | 7 |
| | | | Promoter | |
| Probe 14 | CpG01733570 | 24797656 | Putative | 7 |
| | | | Promoter | |
| Probe 15 | CpG25723149 | 24797680 | Putative | 7 |
| | | | Promoter | |
| Probe 16 | CpG22804000 | 24797691 | Putative | 7 |
| | | | Promoter | |
| Probe 17 | CpG07504598 | 24797786 | Putative | 7 |
| | | | Promoter | |
| Probe 18 | CpG15037663 | 24797835 | Putative | 7 |
| | | | Promoter | |
| Probe 19 | CpG19706795 | 24797839 | Putative | 7 |
| | | | Promoter | |
| Probe 20 | CpG20764575 | 24797884 | Putative | 7 |
| | | | Promoter | |
| Probe 21 | CpG06301139 | 24798175 | Upstream | 7 |
| | | | Region | |
| Probe 22 | CpG26712096 | 24798855 | Upstream | 7 |
| | | | Region | |

Statistical Analyses

We designated the following clinicopathological parameters from the TCGA clinical patient data files with which to perform association analyses: age at diagnosis, gender, ethnicity and pathological tumour stage (I-IV). The statistical software R (version 3.5.2) was used to carry out all the statistical analyses. All reported p-values are two-sided, and those less than or equal to 0.05 were considered statistically significant. To account for the non-independence between measurements from the same individuals, a linear mixed model was fitted and included a random effect for sample barcodes while the significance of the fixed effects was tested via the F-test with a Kenward-Roger correction for the number of degrees of freedom. In all regression models age was accounted for as a covariate, but it was excluded from the final model if its effect on the outcome was not significant. The relation between GSDME methylation and RNA-seq expression was examined using linear regression models, analysis of variance and Spearman's. Associations between methylation and the designated clinicopathological parameters were studied in a similar manner. In all regression models age was accounted for as a covariate, but it was excluded from the final model if its effect on the outcome was not significant.

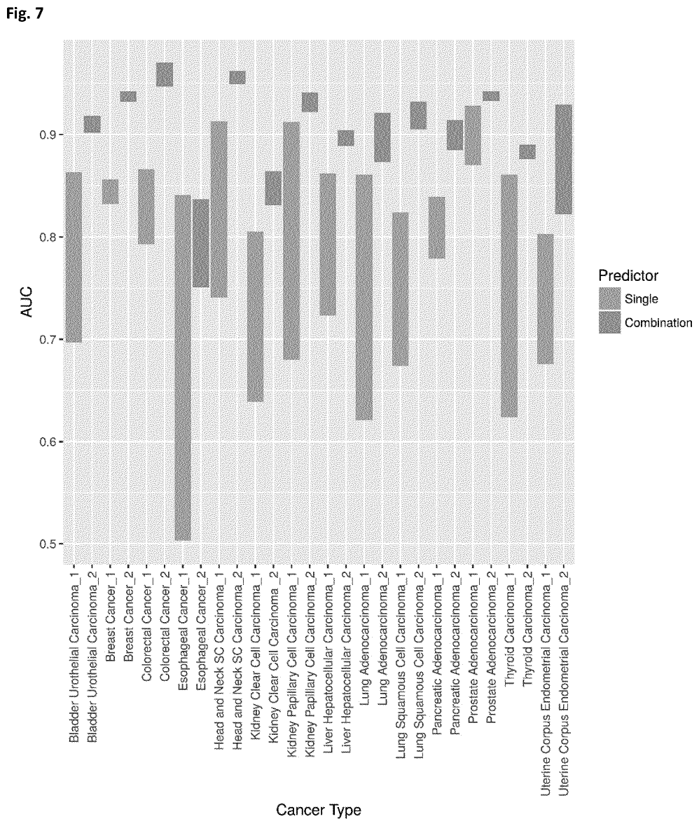

To assess the viability of GSDME methylation as a pan-cancer biomarker a two-fold approach was considered. In a first step, the analysis was carried out on each of the 14 individual datasets. Binary logistic regression models were fitted to predict tissue type (normal/tumour) using different combinations of CpG methylation values as predictors. Stepwise multiple regression was used to determine the best combination of the 22 CpGs. The final model was chosen based on the highest Akaike information criterion (AIC) values with the lowest number of predictors possible. The accuracy of the model predictions was assessed by plotting receiver operating characteristic (ROC) curves. A ten-fold cross validation of these results was then performed. In a second step, we considered each of the 14 datasets individually where we fit binary logistic regression models to all cases and controls in that dataset and calculated model metrics. Based on previous results in the top 5 most common cancer types including breast and colorectal cancers where 2 CpG predictors performed significantly better than only 1 in identifying tissue type, we tried to reach the highest model performance before overfitting (FIG. 7). 3 GSDME probes as model predictors was the optimal number that yielded the best AUC without causing any model overfitting across all 14 datasets and 1540 combinations were tested.

In a second step, we aggregated all the different datasets into one large cohort comprising 719 normal and 5783 tumour samples. We then carried out a similar analysis to the one described above. 719 cases were then chosen at random and considered along with the 719 controls. Binary logistic regression with 10-fold cross validation was then was fitted to predict tissue type (case/control) based on methylation values. The accuracy of the model predictions was assessed by plotting receiver operating characteristic (ROC) curves and calculating the area under the curve (AUC). This process was bootstrapped 1000 times, each with a random selection of cases out of the total pool, and model metrics averaged out. Using the described methodology, we tested all 22 GSDME probes (β-values) individually as model predictors as well as combinations or 2, 3, 4, 5 and 6 probes as predictors.

To test the potential of GSDME methylation as a tumour-specific biomarker, we used the partial least squares-discriminant analysis (PLSDA) algorithm to distinguish between the different cancers. To that end, all 14 datasets were pooled together, resulting in a pooled dataset of 5783 tumours each being 1 of 14 cancer types. The algorithm was run using combinations of 6 probes and ROC curves with AUC values were generated for predicting each cancer type against the 13 others. Moreover, additional Illumina 450K CpG methylation datasets were downloaded from the Gene Expression Omnibus (GEO) database (www.ncbi.nlm.nih.gov/geo/) (GEO accession numbers GSE52865 breast cancer, GSE68060 colorectal cancer, GSE77718 colorectal cancer, GSE89852 hepatocellular cancer, GSE97466 thyroid cancer), and were used for the subsequent external validation. The final model was refit on each of the external datasets and the AUC was recalculated for the new predictions.

The statistical software R (version 3.4.1.) was used to carry out all the statistical analyses. All used p-values were two-sided, and those less than or equal to 0.05 were considered statistically significant.

### Results

GSDME Differential Methylation Across 14 Tumour Types

To comprehensively explore the methylation patterns of GSDME, we investigated differential methylation in 14 different tumours, by comparing cancer samples with corresponding normal tissue at a distance from the tumour. We found differential methylation of GSDME CpGs in all 14 cancer types, ranging from 6/22 CpGs in kidney, pancreatic and thyroidal cancers, to 22/22 CpGs in breast and colorectal cancers. Differential methylation was greatly variable amongst the cancer types, on average 13 out of the 22 CpG probes were differentially methylated between tumour and normal tissues (P=3.107 E-30 to 4.96 E-2) (data not shown). No significant correlation was found between the number of differentially methylated probes and dataset sizes (Pearson's correlation p-value>0.05).

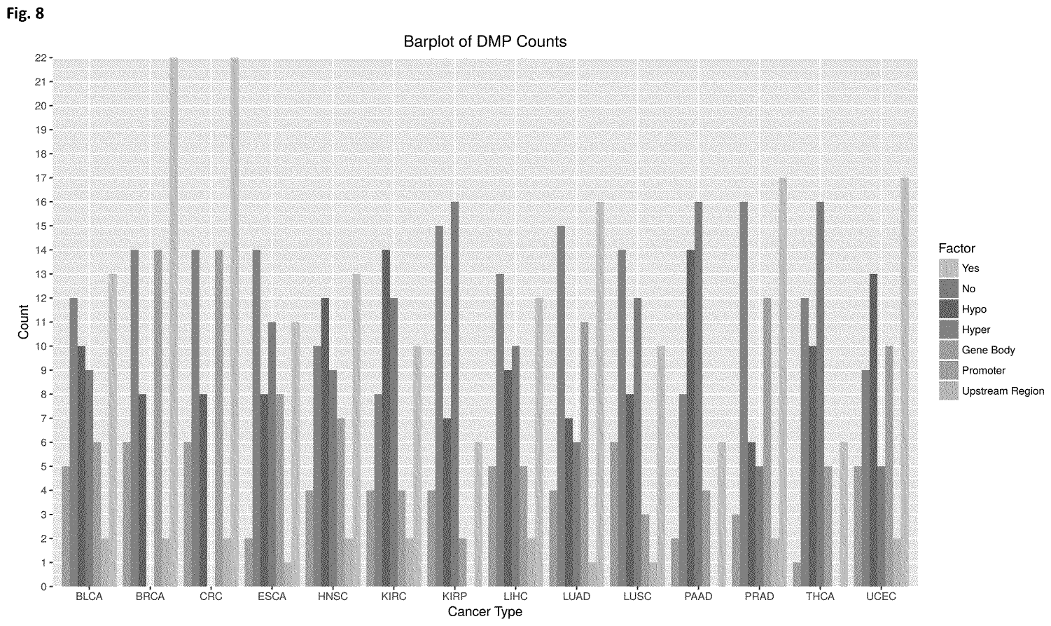

On average, differentially methylated probes (DMPs) were more frequently detected in the putative promoter region, with less DMPs being in the putative gene body and upstream regions. In general, we found CpGs to be hyper-methylated in the tumour samples as opposed to the control sample, especially those located with the putative GSDME promoter region (FIG. 8).

A correlation matrix for the methylation values of all 22 CpGs (in colorectal and breast cancers) was constructed to investigate the association between the methylation of different regions in the GSDME gene. This exhibited a block-like clustering; a smaller cluster made up of the six CpGs located in the gene body, and a larger cluster made up of the remaining 14 CpGs located in the putative gene promoter region (already shown in FIG. 4. Additionally, the last two CpGs located upstream of the putative gene promoter region clustered together and had a pattern similar to the gene body cluster. In these clusters, the larger CpG group, pertaining to probes in the putative promoter region, had the largest positive pairwise correlation coefficients whereas the smaller group had lower positive coefficients, all of which having significant p-values less than 0.05 (FIG. 4).

Correlation coefficients are indicated by circle color and size. All correlation coefficients had a p-value greater than 0.05. Two distinct clusters can be seen based on the correlation coefficients of the methylation values; promoter region CpGs form the biggest cluster (14 out of 22) while gene body CpGs for the smaller cluster, CpG21 and CpG22 cluster together and follow closely the pattern of the intragenic CpGs.

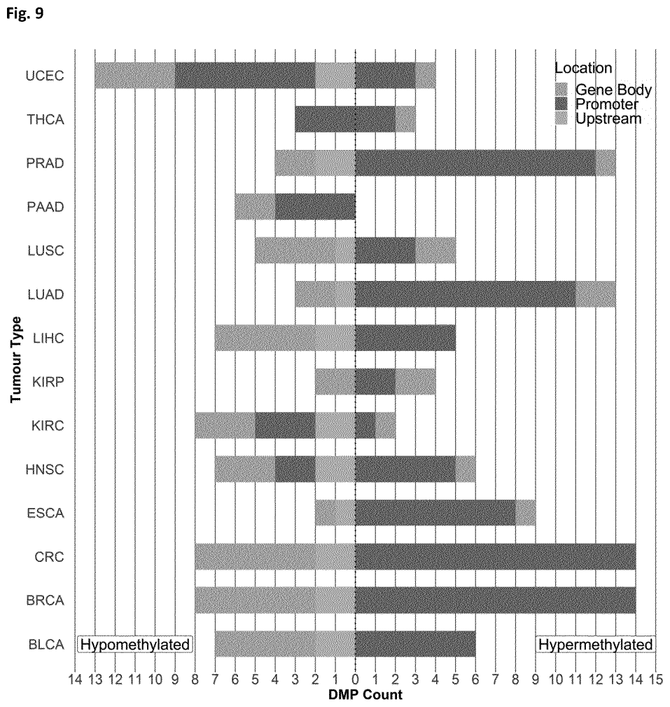

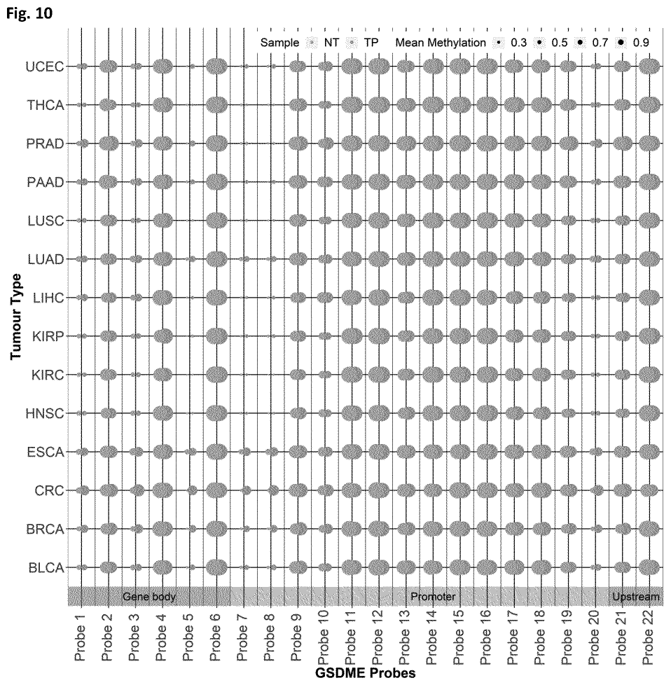

In the breast and colorectal cancer datasets all 22 GSDME CpGs were differentially methylated, while the kidney, pancreatic and thyroid tumours exhibited differential methylation in only six CpGs (FIG. 9). In general, those differentially methylated probes were hypomethylated in the normal tissue compared to the tumour tissues. Uterine carcinomas reported the highest count of hypomethylated GSDME CpGs, followed by breast, colorectal, and renal clear cell tumours, while breast and colorectal tumours followed by lung and prostate tumours had the highest count of hypermethylated CpGs (FIG. 9-10). Interestingly, differential methylation was not limited to promoter CpGs. In all of the tumour types investigated, one or more of the six intragenic probes were differentially methylated. Even probes in the region upstream of the promoter, which follow methylation patterns of gene body CpGs, were differentially methylated in 11 out of the 14 tumours (FIG. 9-10).

GSDME Methylation as a Pan-Cancer Detection Biomarker

Initial Predictor Combination Selection

We used binary logistic regression to identify combinations of GSDME probes that could be used to differentiate tumour from normal samples across the different cancer types. In accordance with other studies on TCGA data, we only chose datasets that had a tumour to normal sample ratio of 10% or a minimum of ten tumour-normal pairs. Next, we pooled the 14 different tumour datasets resulting in 719 and 5783 normal and tumour samples respectively. We regressed binary models with combinations of one to six methylation probes as predictors and bootstrapped these calculations 1,000 times each to avoid the case-to-control imbalance in the dataset. In total 110,056 combinations were tested, of which 74,613 comprised six probes. The average area under the curve (AUC) was 0.627 using only a single probe, while it was 0.871 using a combination of six probes (Table 4). Using combinations of seven or more probes, we encountered model overfitting with diminishing returns, considering the major increase in the number of combinations to test, with only minimal improvements in AUC. Single probes were less than optimal for discrimination between cases and controls, the best of which, probe 6, scored an AUC of 0.737 while the rest had AUCs in the 0.60 s range. While relevant, these findings are unsurprising as information obtained from only one predictor is too little to make a clear distinction given the considerable heterogeneity of the samples and the inherent diversity between the different tumours. Another factor involved in these interpretations is the narrow dynamic range associated with the Beta-value which only extends from 0 to 1, thus limiting the size of discernible differences at one single position. In contrast, models employing combinations of five to six probes as predictors performed exceptionally well across the cancer types, with AUCs reaching 0.862 and 0.871 respectively. The combination of probes with the best predictive power included probes 3, 12, 14, 18, 20 and 21. Of these probes, one is in the putative gene body region, four are in the promoter and one is present in the upstream region (FIG. 10 and Table 1 and 3). The top scoring combinations also included the mentioned probes in addition to the promoter probes 11, 13 and 19 in an array of combinations.

TABLE 4
Average AUCs of the different CpG predictor combinations

| No. CpGs | Number of tested | Average |
|---|---|---|
| predictors | combinations | AUC |
| 1 | 22 | 0.627 |
| 2 | 231 | 0.795 |
| 3 | 1540 | 0.820 |
| 4 | 7315 | 0.840 |
| 5 | 26335 | 0.861 |
| 6 | 75613 | 0.871 |

Individual Dataset Analysis

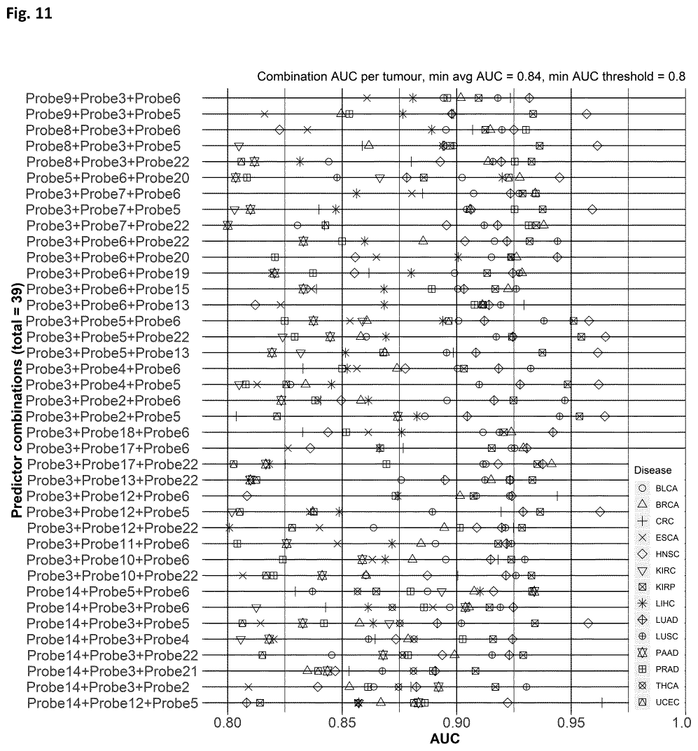

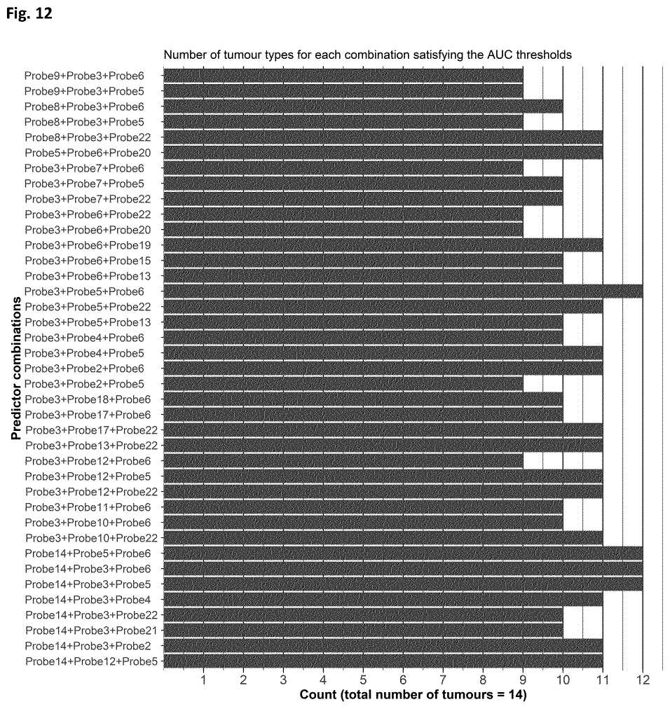

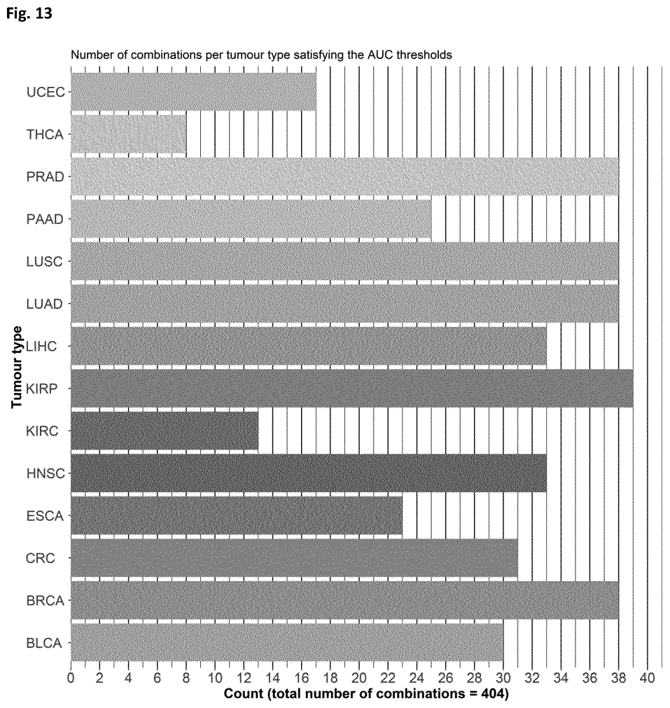

To ensure that dataset sample size did not cause any bias for the selection model in the pooled dataset, we then reproduced the same analysis in the 14 individual datasets separately. For these combinations to possess pan-cancer functionality, they must i) present consistently high AUCs across the different datasets with a relatively small standard deviation, and ii) larger datasets should not be correlated with better AUCs. Single probes performed better on average in the individual datasets with an AUC of 0.810. This can be attributed to the smaller sample of these datasets and the decrease in heterogeneity amongst the two sample classes. A total of 1540 different combinations of three probes (more than three predictors resulted in model overfitting) were tested with varying AUC outcomes ranging from 0.520 to 0.974. No discernible effect of sample size on AUC was observed. In order to combine the results from both analyses and select for the best performing probe combinations, we set two filters. For both the pooled and individual analyses, we set the minimum average AUC in bins of 0.1 increments, starting at 0.80 and ending at maximal AUC. Additionally, for the individual analysis the minimum threshold for any probe combination should not be below 0.80 (FIG. 11) (Table 5). This resulted in 14 combinations, with an AUC of 0.85 or more, in the pooled analysis: in this scenario the top recurring probes to these combinations were probes 4, 6, and 16. In the individual analysis, 7 combinations fit the two filters and the top recurring probes to these combinations were probes 4, 14, and 16. Thirty nine combinations of 3 probes satisfied the 0.84 AUC filter with several demonstrating AUCs above 0.90 for breast, colorectal, prostate, kidney and lung cancers (FIG. 11), which are amongst the most common cancer types worldwide. Four combinations that included probes 3, 5, 6, and 14 satisfied the set filters in 12 of the 14 tumour types, followed by 14 others in 11 of the 14 types (FIG. 12). Kidney tumours, followed by pancreatic, prostate, lung and breast had the highest number of probe combinations that satisfied the set filters at 39 and 38 combinations respectively (FIG. 13).

Final Model and Validation

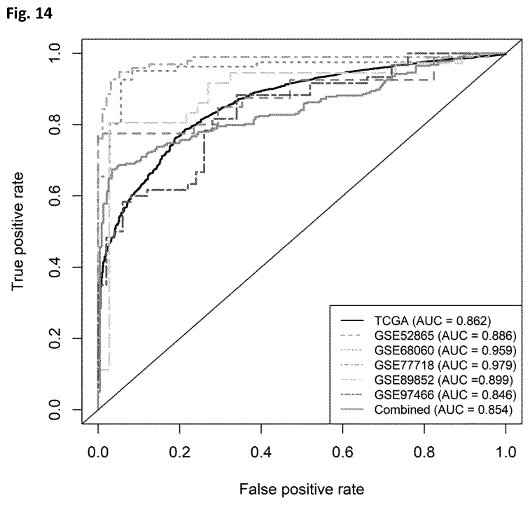

The top six probes from the pooled analysis (probes 3, 12, 14, 18, 20 and 21,) were then selected for further model construction and validation. A logistic regression model was implemented based on the selected six features and trained on the pooled dataset involving the 14 tumour types (N=6502). This logistic regression model achieved a 10-fold cross validated AUC of 0.86 in the training set (FIG. 14). Applying a 0.55 cut-off value, the sensitivity, specificity and overall accuracy were 98.8%, 94.2% and 89.7% respectively. We then independently validated the constructed model using five external datasets downloaded from the Gene Expression Omnibus (GEO) (GSE52865 breast cancer, GSE68060 colorectal cancer, GSE77718 colorectal cancer, GSE89852 hepatocellular cancer, GSE97466 thyroid cancer), as well as a pooled dataset of the five. The AUCs for those five independent datasets were 0.89, 0.96, 0.97, 0.90, 0.85 respectively, and 0.85 for the pooled set (FIG. 14). To assess the homogeneity of the relationship between CpG methylation and sample type, we included tissue type as dependent variable, and added CpG methylation, stage and the interaction between methylation and stage as independent variables in the logistic regression model. We then tested the significance of the interaction term using a likelihood ratio test, comparing the fit of the model with both main effects and their interaction term, against the model with only the main effects of methylation and stage. We did not find any significant effects of disease stage or age on tissue type prediction and thus concluded that methylation was not significantly altered by stage. In all, the six-probe model demonstrated a good predictive power in a pan-cancer setting, and its consistent performance in external datasets shows its validity as a detection marker.

GSDME Methylation as a Tumour-Specific Biomarker

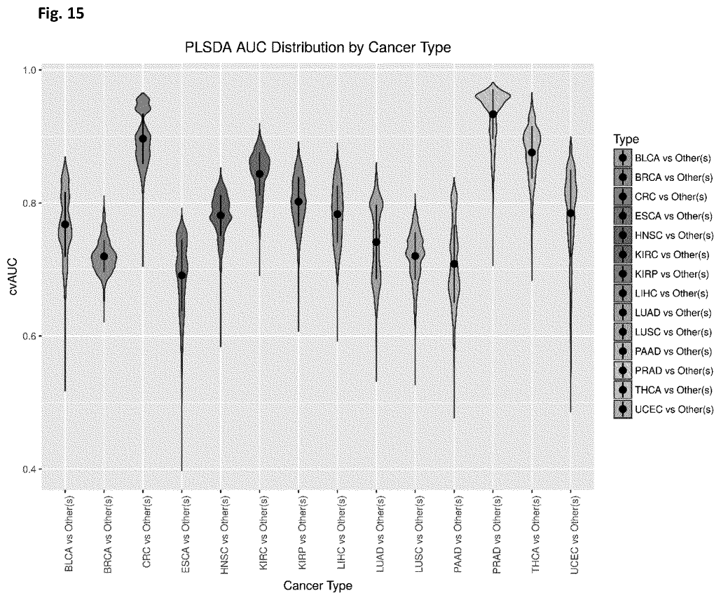

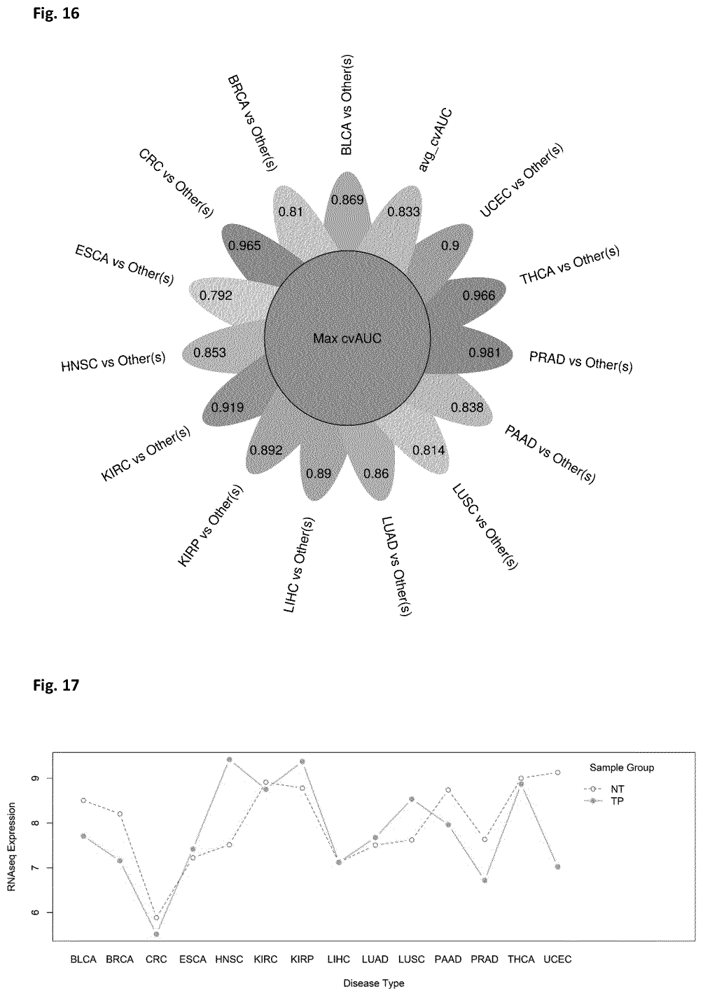

We explored the capacity of GSDME methylation to differentiate between different tumour types based on combinations of CpG probes. We again decided on combinations of six probes, as preliminary testing showed the highest average AUC with the least number of predictors and the most reasonable number of combinations to test. We used the Partial Least Squares Discriminant Analysis (PLSDA) to fit models for 74,613 combinations using a pooled dataset of tumours across the 14 types (N=5783). PLSDA is well suited for multi-class predictive modeling, works well with large datasets and has demonstrated merit in medical diagnostics. The average cross-validated AUC for classifying the 14 tumour types was 0.833 and was achieved using probes 5, 7, 11, 16,18 and 22. A large portion of combinations performed well in detecting colorectal, kidney, prostate and thyroid tumours with local AUC means above the 0.80 mark (FIG. 15-16). Other tumour types showed a wider spread in AUCs with lower means; however, the local AUC maxima were all 0.80 or above (FIG. 15). Prostate cancer could be discriminated with the highest power against all other tumours (AUC=0.981) followed by thyroid (AUC=0.966), colorectal (AUC=0.965) and kidney (AUC=0.919) cancers. Esophageal tumours were the most problematical to discriminate amongst the tumour types with an AUC of 0.792, which is still acceptable in a prediction setting (FIG. 16). The average max prediction AUC value across the different datasets was 0.833 with prostate, thyroidal colorectal, uterine and kidney cancers scoring AUCs of 0.900 or higher (FIG. 16). Using the 0.833 average AUC as a cutoff point, 15 CpG predictor combinations can be retained (FIG. 8), these combinations included CpGs cg09333471 (CpG7), cg17569154 (CpG5), cg15037663 (CpG6), cg07293520 (CpG11), cg26712096 (CpG22) and cg25723149 (CpG15).

The best performing combinations for all the predictions included probes 3, 5, 7, 14, 19 and 22 which comprised all three regions of the GSDME gene and were not limited to the promoter region where the greatest variations in methylation would typically be expected.

In all, using different combinations of up to 6 CpG probes located in the GSDME gene allowed us to construct a highly robust model that could accurately distinguish between normal and cancer tissue, and between 14 different cancer types, based on methylation values. Although some combinations may have lower AUCs in one setting or the other, using different combinations ensures that one or more have a high enough accuracy and precision to make this approach valid for application in a liquid biopsies setting. Using the twofold approach and bootstrapping testing for the pan-cancer marker, safeguards against an overly positive classifier model and ensure that the resulting high AUCs are not due to any class imbalances in the dataset. The exceptional in silico performance of the thoroughly identified CpG dinucleotides in a large patient cohort, makes this study a stepping stone towards developing a biomarker assay for the detection of cancer, in the context of liquid biopsy-based assay. Another novelty is the model's ability to accurately distinguish between the different disease types, this could have important implications on clinical cancer diagnosis.

The Relation of GSDME Methylation to RNA-Seq Expression and Clinicopathological Parameters

We examined GSDME expression levels using RNA-seq data downloaded from TCGA. The mean expression in normal tissues was 7.99 while it was slightly lower in tumour tissues at 7.80, but these differences were not significant. In general, higher expression levels could be observed in the normal tissues as compared to the tumours, the only exception were head and neck, kidney, esophageal, lung and liver tumours (Table 6, FIG. 17). Contrary to the general dogma, we could not find a very significant effect of methylation on RNA-seq expression in GSDME. On average the methylation 5 of the 22 probes was significantly associated with RNA-seq expression, and the methylation of 3 probes on average per tumour type showed an association with gene expression. Head and neck as well as kidney renal papillary carcinomas showed a significant association between the methylation of 9 GSDME probes with gene expression, whereas pancreatic cancer showed an association only in 2 probes. Probe 22 exhibited associations across the most tumour types (10 types), while probe 19 did not show any association between methylation and expression levels in any of the cancer types. In general, the significant associations had negative slopes indicating an inverse relationship between methylation and expression, however these slopes were not very large, hence their true effect is still questionable. Moreover; there is no clear association between GSDME expression and methylation as these relations were not ubiquitously significant across promoter or gene body probes, in the majority of tumour types (Table 8). We also analyzed the effect of clinicopathological parameters, namely age at diagnosis, gender, and ethnicity, on the methylation of GSDME, using linear models. Although some of the p-values were lower than the significant p-value, their corresponding slopes were almost at 0 hence their effect on methylation is negligible (Table 8).

TABLE 5
Table of the individual dataset analysis AUC values that satisfy the specified threshold (minimum average AUC = 0.84
and minimum AUC threshold = 0.80. NA values are tumour types for which the probe combination did not meet the AUC thresholds).

| Probe | | | | | | | | | | | | | | |
|---|---|---|---|---|---|---|---|---|---|---|---|---|---|---|
| Combination | BLCA | BRCA | CRC | ESCA | HNSC | KIRC | KIRP | LIHC | LUAD | LUSC | PAAD | PRAD | THCA | UCEC |
| cg00473134 + | NA | 0.86 | 0.86 | NA | 0.96 | 0.80 | 0.94 | 0.89 | 0.89 | 0.90 | NA | 0.90 | NA | NA |
| cg04317854 + | | | | | | | | | | | | | | |
| cg17569154 | | | | | | | | | | | | | | |
| cg00473134 + | 0.90 | 0.91 | 0.91 | 0.83 | 0.82 | NA | 0.91 | 0.89 | 0.92 | 0.92 | NA | 0.93 | NA | NA |
| cg04317854 + | | | | | | | | | | | | | | |
| cg19260663 | | | | | | | | | | | | | | |
| cg00473134 + | 0.84 | 0.91 | 0.88 | NA | 0.89 | NA | 0.93 | 0.83 | 0.92 | 0.92 | 0.81 | 0.93 | NA | 0.81 |
| cg04317854 + | | | | | | | | | | | | | | |
| cg26712096 | | | | | | | | | | | | | | |
| cg01733570 + | NA | 0.83 | 0.85 | NA | 0.85 | NA | 0.91 | NA | 0.89 | 0.87 | 0.84 | 0.89 | 0.88 | 0.84 |
| cg04317854 + | | | | | | | | | | | | | | |
| cg06301139 | | | | | | | | | | | | | | |
| cg01733570 + | NA | 0.88 | 0.86 | 0.82 | 0.87 | 0.81 | 0.92 | NA | 0.92 | 0.86 | 0.82 | 0.90 | 0.88 | NA |
| cg04317854 + | | | | | | | | | | | | | | |
| cg12922093 | | | | | | | | | | | | | | |
| cg01733570 + | 0.86 | 0.85 | 0.88 | 0.81 | 0.84 | NA | 0.92 | NA | 0.88 | 0.93 | 0.89 | 0.86 | 0.87 | NA |
| cg04317854 + | | | | | | | | | | | | | | |
| cg14205998 | | | | | | | | | | | | | | |
| cg01733570 + | NA | 0.86 | NA | 0.81 | 0.96 | 0.87 | 0.93 | 0.86 | 0.89 | 0.90 | 0.83 | 0.84 | 0.88 | 0.81 |
| cg04317854 + | | | | | | | | | | | | | | |
| cg17569154 | | | | | | | | | | | | | | |
| cg01733570 + | 0.90 | 0.91 | 0.84 | 0.89 | NA | 0.81 | 0.91 | 0.86 | 0.92 | 0.92 | 0.90 | 0.89 | 0.87 | NA |
| cg04317854 + | | | | | | | | | | | | | | |
| cg19260663 | | | | | | | | | | | | | | |
| cg01733570 + | 0.85 | 0.90 | NA | NA | 0.89 | NA | 0.93 | NA | 0.92 | 0.92 | 0.87 | 0.88 | 0.88 | 0.82 |
| cg04317854 + | | | | | | | | | | | | | | |
| cg26712096 | | | | | | | | | | | | | | |
| cg01733570 + | NA | 0.87 | 0.96 | NA | 0.92 | 0.86 | 0.81 | 0.86 | 0.81 | NA | 0.88 | 0.89 | 0.86 | 0.88 |
| cg04770504 + | | | | | | | | | | | | | | |
| cg17569154 | | | | | | | | | | | | | | |
| cg01733570 + | 0.89 | 0.91 | 0.83 | NA | 0.92 | 0.89 | 0.86 | 0.91 | NA | 0.84 | 0.93 | 0.88 | 0.86 | 0.93 |
| cg17569154 + | | | | | | | | | | | | | | |
| cg19260663 | | | | | | | | | | | | | | |
| cg03995857 + | NA | 0.85 | 0.90 | 0.82 | 0.96 | NA | 0.93 | 0.88 | 0.90 | 0.90 | NA | 0.85 | NA | NA |
| cg04317854 + | | | | | | | | | | | | | | |
| cg17569154 | | | | | | | | | | | | | | |
| cg03995857 + | 0.89 | 0.90 | 0.92 | 0.86 | NA | NA | 0.91 | 0.88 | 0.93 | 0.92 | NA | 0.90 | NA | NA |
| cg04317854 + | | | | | | | | | | | | | | |
| cg19260663 | | | | | | | | | | | | | | |
| cg04317854 + | NA | 0.84 | 0.92 | 0.84 | 0.96 | 0.80 | 0.94 | 0.85 | 0.93 | 0.89 | NA | 0.84 | NA | 0.81 |
| cg04770504 + | | | | | | | | | | | | | | |
| cg17569154 | | | | | | | | | | | | | | |
| cg04317854 + | 0.91 | 0.90 | 0.94 | NA | 0.81 | NA | 0.91 | 0.87 | 0.92 | 0.92 | NA | 0.87 | NA | NA |
| cg04770504 + | | | | | | | | | | | | | | |
| cg19260663 | | | | | | | | | | | | | | |
| cg04317854 + | 0.86 | 0.89 | 0.92 | 0.84 | 0.91 | NA | 0.93 | 0.80 | 0.92 | 0.92 | NA | 0.90 | NA | 0.83 |
| cg04770504 + | | | | | | | | | | | | | | |
| cg26712096 | | | | | | | | | | | | | | |
| cg04317854 + | 0.89 | 0.88 | 0.92 | 0.85 | NA | NA | 0.92 | 0.87 | 0.92 | 0.92 | 0.83 | 0.80 | NA | NA |
| cg07293520 + | | | | | | | | | | | | | | |
| cg19260663 | | | | | | | | | | | | | | |
| cg04317854 + | 0.90 | 0.88 | 0.92 | 0.86 | NA | NA | 0.92 | 0.87 | 0.91 | 0.93 | 0.86 | 0.82 | NA | NA |
| cg07320646 + | | | | | | | | | | | | | | |
| cg19260663 | | | | | | | | | | | | | | |
| cg04317854 + | 0.86 | 0.86 | 0.90 | 0.81 | 0.89 | NA | 0.93 | NA | 0.92 | 0.93 | 0.84 | 0.82 | NA | 0.82 |
| cg07320646 + | | | | | | | | | | | | | | |
| cg26712096 | | | | | | | | | | | | | | |
| cg04317854 + | 0.92 | 0.93 | 0.88 | 0.83 | 0.84 | NA | 0.92 | 0.87 | 0.93 | 0.93 | NA | 0.87 | NA | NA |
| cg07504598 + | | | | | | | | | | | | | | |
| cg19260663 | | | | | | | | | | | | | | |
| cg04317854 + | 0.91 | 0.94 | 0.83 | NA | 0.92 | NA | 0.94 | 0.82 | 0.94 | 0.91 | 0.82 | 0.87 | NA | 0.80 |
| cg07504598 + | | | | | | | | | | | | | | |
| cg26712096 | | | | | | | | | | | | | | |
| cg04317854 + | NA | 0.91 | 0.84 | NA | 0.96 | 0.80 | 0.94 | 0.85 | 0.91 | 0.90 | 0.81 | 0.93 | NA | NA |
| cg09333471 + | | | | | | | | | | | | | | |
| cg17569154 | | | | | | | | | | | | | | |
| cg04317854 + | 0.91 | 0.93 | 0.89 | 0.88 | NA | NA | 0.93 | 0.86 | 0.92 | 0.93 | NA | 0.93 | NA | NA |
| cg09333471 + | | | | | | | | | | | | | | |
| cg19260663 | | | | | | | | | | | | | | |
| cg04317854 + | 0.83 | 0.94 | 0.84 | NA | 0.90 | NA | 0.93 | NA | 0.92 | 0.91 | 0.80 | 0.93 | NA | 0.84 |
| cg09333471 + | | | | | | | | | | | | | | |
| cg26712096 | | | | | | | | | | | | | | |
| cg04317854 + | 0.83 | 0.83 | NA | 0.81 | 0.96 | 0.80 | 0.95 | 0.85 | 0.93 | 0.91 | NA | 0.81 | NA | 0.83 |
| cg12922093 + | | | | | | | | | | | | | | |
| cg17569154 | | | | | | | | | | | | | | |
| cg04317854 + | 0.90 | 0.87 | 0.83 | 0.86 | 0.88 | NA | 0.90 | 0.85 | 0.92 | 0.93 | NA | 0.85 | NA | NA |
| cg12922093 + | | | | | | | | | | | | | | |
| cg19260663 | | | | | | | | | | | | | | |
| cg04317854 + | 0.89 | NA | 0.80 | NA | 0.96 | NA | 0.95 | 0.88 | 0.90 | 0.94 | 0.87 | NA | NA | 0.82 |
| cg14205998 + | | | | | | | | | | | | | | |
| cg17569154 | | | | | | | | | | | | | | |
| cg04317854 + | 0.90 | 0.86 | 0.84 | 0.84 | 0.85 | NA | 0.92 | 0.86 | 0.92 | 0.95 | 0.82 | 0.84 | NA | NA |
| cg14205998 + | | | | | | | | | | | | | | |
| cg19260663 | | | | | | | | | | | | | | |
| cg04317854 + | 0.91 | 0.92 | 0.83 | 0.86 | 0.84 | NA | 0.92 | 0.88 | 0.94 | 0.92 | NA | 0.85 | NA | NA |
| cg15037663 + | | | | | | | | | | | | | | |
| cg19260663 | | | | | | | | | | | | | | |
| cg04317854 + | 0.90 | 0.86 | NA | 0.85 | 0.96 | 0.86 | 0.95 | 0.89 | 0.91 | 0.94 | 0.84 | 0.82 | NA | 0.90 |
| cg17569154 + | | | | | | | | | | | | | | |
| cg19260663 | | | | | | | | | | | | | | |
| cg04317854 + | NA | 0.87 | 0.90 | NA | 0.96 | 0.83 | 0.94 | 0.85 | 0.91 | 0.90 | 0.82 | 0.87 | NA | NA |
| cg17569154 + | | | | | | | | | | | | | | |
| cg24805239 | | | | | | | | | | | | | | |
| cg04317854 + | 0.86 | 0.86 | NA | NA | 0.96 | 0.82 | 0.95 | 0.87 | 0.92 | 0.92 | 0.84 | 0.83 | NA | 0.92 |
| cg17569154 + | | | | | | | | | | | | | | |
| cg26712096 | | | | | | | | | | | | | | |
| cg04317854 + | 0.90 | 0.93 | 0.86 | 0.82 | 0.86 | NA | 0.91 | 0.88 | 0.92 | 0.93 | 0.82 | 0.84 | NA | NA |
| cg19260663 + | | | | | | | | | | | | | | |
| cg19706795 | | | | | | | | | | | | | | |
| cg04317854 + | 0.92 | 0.93 | NA | 0.86 | 0.86 | NA | 0.92 | 0.90 | 0.94 | 0.92 | NA | 0.82 | NA | NA |
| cg19260663 + | | | | | | | | | | | | | | |
| cg20764575 | | | | | | | | | | | | | | |
| cg04317854 + | 0.91 | 0.91 | 0.93 | 0.82 | 0.81 | NA | 0.91 | 0.87 | 0.91 | 0.92 | NA | 0.91 | NA | NA |
| cg19260663 + | | | | | | | | | | | | | | |
| cg24805239 | | | | | | | | | | | | | | |
| cg04317854 + | 0.90 | 0.92 | 0.84 | 0.84 | NA | NA | 0.92 | 0.87 | 0.90 | 0.93 | 0.83 | 0.89 | NA | NA |
| cg19260663 + | | | | | | | | | | | | | | |
| cg25723149 | | | | | | | | | | | | | | |
| cg04317854 + | 0.92 | 0.89 | NA | NA | 0.90 | NA | 0.93 | 0.86 | 0.92 | 0.94 | 0.83 | 0.85 | NA | NA |
| cg19260663 + | | | | | | | | | | | | | | |
| cg26712096 | | | | | | | | | | | | | | |
| cg04317854 + | 0.88 | 0.91 | 0.89 | NA | 0.89 | NA | 0.93 | 0.81 | 0.92 | 0.91 | 0.81 | 0.92 | NA | 0.81 |
| cg24805239 + | | | | | | | | | | | | | | |
| cg26712096 | | | | | | | | | | | | | | |
| cg17569154 + | 0.90 | 0.93 | NA | NA | 0.94 | 0.87 | 0.89 | 0.92 | 0.88 | 0.85 | 0.80 | 0.81 | NA | 0.92 |
| cg19260663 + | | | | | | | | | | | | | | |
| cg20764575 | | | | | | | | | | | | | | |

TABLE 6
Summary of the RNA-seq expression levels in the different
sample types and across the different tumour types

| Sample | Tumour | | | |
|---|---|---|---|---|
| Type | Type | N | Mean | SD |
| NT | BLCA | 19 | 8.506 | 0.982 |
| NT | BRCA | 113 | 8.206 | 0.448 |
| NT | CRC | 51 | 5.885 | 1.213 |
| NT | ESCA | 11 | 7.226 | 1.220 |
| NT | HNSC | 44 | 7.516 | 0.830 |
| NT | KIRC | 72 | 8.914 | 0.405 |
| NT | KIRP | 32 | 8.781 | 0.510 |
| NT | LIHC | 50 | 7.122 | 0.558 |
| NT | LUAD | 59 | 7.507 | 0.731 |
| NT | LUSC | 51 | 7.623 | 0.544 |
| NT | PAAD | 4 | 8.740 | 1.095 |
| NT | PRAD | 52 | 7.636 | 0.715 |
| NT | THCA | 59 | 9.004 | 0.379 |
| NT | UCEC | 24 | 9.132 | 0.766 |
| TP | BLCA | 408 | 7.709 | 1.854 |
| TP | BRCA | 1095 | 7.156 | 0.852 |
| TP | CRC | 379 | 5.515 | 1.494 |
| TP | ESCA | 184 | 7.419 | 2.023 |
| TP | HNSC | 520 | 9.422 | 1.275 |
| TP | KIRC | 533 | 8.753 | 0.945 |
| TP | KIRP | 290 | 9.376 | 1.169 |
| TP | LIHC | 371 | 7.121 | 1.799 |
| TP | LUAD | 515 | 7.676 | 1.315 |
| TP | LUSC | 502 | 8.536 | 1.302 |
| TP | PAAD | 178 | 7.963 | 1.037 |
| TP | PRAD | 497 | 6.718 | 0.868 |
| TP | THCA | 505 | 8.873 | 0.674 |
| TP | UCEC | 176 | 7.021 | 1.211 |

TP = primary tumor,
NT = normal tissue;
N = sample size;
SD = standard deviation

TABLE 7
Table of the linear regression results for the analysis of RNA-seq expression and
methylation. Numbers followed by an asterisk (*) represent significant p-values.

| | | BLCA_p- | | BRCA_p- | | CRC_p- | |
|---|---|---|---|---|---|---|---|
| probes | BLCA_slope | value | BRCA_slope | value | CRC_slope | value | ESCA_slope |
| cg00473134 | 0.087 | 0.848 | 0.005 | 0.986 | 0.044 | 0.929 | −0.182 |
| cg01733570 | 1.498 | 0.021* | −0.063 | 0.850 | 0.304 | 0.523 | 2.181 |
| cg03995857 | 2.121 | 0.000* | 0.373 | 0.182 | −1.684 | 0.011* | 0.548 |
| cg04317854 | 1.027 | 0.131 | −0.133 | 0.650 | −0.366 | 0.422 | 1.493 |
| cg04770504 | −0.533 | 0.155 | 0.048 | 0.828 | 0.385 | 0.358 | −0.492 |
| cg06301139 | 0.932 | 0.106 | 0.794 | 0.009* | 0.646 | 0.220 | 0.046 |
| cg07293520 | −1.864 | 0.030* | −0.365 | 0.145 | −0.703 | 0.269 | −3.696 |
| cg07320646 | −2.580 | 0.020* | 0.183 | 0.519 | −0.679 | 0.238 | 1.695 |
| cg07504598 | −0.206 | 0.846 | −0.108 | 0.679 | −0.385 | 0.449 | −1.136 |
| cg09333471 | 0.995 | 0.287 | 0.245 | 0.377 | −0.569 | 0.285 | −2.871 |
| cg12922093 | 0.075 | 0.949 | −0.113 | 0.753 | 0.781 | 0.184 | 0.986 |
| cg14205998 | −0.067 | 0.951 | 0.563 | 0.114 | 1.424 | 0.076 | 5.632 |
| cg15037663 | 0.954 | 0.395 | −0.353 | 0.307 | −0.350 | 0.667 | −1.620 |
| cg17569154 | −1.132 | 0.088 | 0.371 | 0.162 | 0.485 | 0.468 | 2.400 |
| cg17790129 | −1.823 | 0.095 | −0.951 | 0.025* | −0.962 | 0.287 | −0.902 |
| cg19260663 | 1.146 | 0.316 | 0.130 | 0.736 | −0.397 | 0.616 | −6.030 |
| cg19706795 | −1.087 | 0.072 | −0.254 | 0.399 | 0.633 | 0.202 | −0.113 |
| cg20764575 | 0.436 | 0.596 | 0.489 | 0.112 | −0.354 | 0.568 | 2.244 |
| cg22804000 | −0.205 | 0.870 | −0.093 | 0.830 | 0.334 | 0.684 | −0.316 |
| cg24805239 | 0.403 | 0.637 | −0.362 | 0.258 | −2.086 | 0.021* | 0.937 |
| cg25723149 | 1.277 | 0.146 | −0.085 | 0.792 | 0.746 | 0.180 | −2.661 |
| cg26712096 | 1.136 | 0.034* | 0.667 | 0.019* | 2.781 | 0.000* | 1.357 |

| | ESCA_p- | | HNSC_p- | | KIRC_p- | | KIRP_p- |
|---|---|---|---|---|---|---|---|
| probes | value | HNSC_slope | value | KIRC_slope | value | KIRP_slope | value |
| cg00473134 | 0.813 | −1.527 | 0.038* | 0.146 | 0.783 | −0.205 | 0.769 |
| cg01733570 | 0.101 | 3.180 | 0.037* | 0.945 | 0.583 | 6.252 | 0.033* |
| cg03995857 | 0.558 | 0.643 | 0.367 | 0.738 | 0.003* | 1.538 | 0.000* |
| cg04317854 | 0.175 | 2.541 | 0.001* | −0.356 | 0.620 | −3.556 | 0.010* |
| cg04770504 | 0.388 | −1.578 | 0.000* | −1.823 | 0.000* | −0.802 | 0.003* |
| cg06301139 | 0.970 | 1.274 | 0.262 | 0.163 | 0.877 | −0.445 | 0.630 |
| cg07293520 | 0.021* | 0.048 | 0.960 | 0.293 | 0.699 | −3.137 | 0.002* |
| cg07320646 | 0.313 | −6.696 | 0.004* | −1.751 | 0.406 | 5.594 | 0.019* |
| cg07504598 | 0.674 | 6.051 | 0.013* | −4.141 | 0.065 | −6.629 | 0.012* |
| cg09333471 | 0.331 | −4.243 | 0.048* | 7.867 | 0.292 | 1.514 | 0.396 |
| cg12922093 | 0.631 | 1.286 | 0.413 | −9.407 | 0.114 | −4.466 | 0.468 |
| cg14205998 | 0.132 | −0.150 | 0.955 | −3.402 | 0.370 | 9.273 | 0.250 |
| cg15037663 | 0.511 | −0.221 | 0.914 | 2.028 | 0.208 | −6.609 | 0.022* |
| cg17569154 | 0.057 | 0.312 | 0.753 | −0.273 | 0.538 | 0.664 | 0.276 |
| cg17790129 | 0.673 | −1.314 | 0.366 | −2.581 | 0.000* | 1.891 | 0.049* |
| cg19260663 | 0.005* | −2.180 | 0.161 | 2.176 | 0.003* | −1.942 | 0.063 |
| cg19706795 | 0.914 | 0.338 | 0.544 | 0.511 | 0.259 | 0.245 | 0.601 |
| cg20764575 | 0.133 | 0.492 | 0.528 | 0.633 | 0.360 | 0.855 | 0.219 |
| cg22804000 | 0.870 | 0.600 | 0.535 | −0.152 | 0.830 | −1.327 | 0.139 |
| cg24805239 | 0.523 | 0.124 | 0.875 | −0.313 | 0.337 | −0.459 | 0.404 |
| cg25723149 | 0.027* | −2.412 | 0.000* | −2.560 | 0.012* | −4.202 | 0.076 |
| cg26712096 | 0.120 | 3.547 | 0.000* | 3.766 | 0.007* | 1.169 | 0.454 |

| | | LIHC_p- | | LUAD_p- | | LUSC_p- | |
|---|---|---|---|---|---|---|---|
| probes | LIHC_slope | value | LUAD_slope | value | LUSC_slope | value | PAAD_slope |
| cg00473134 | −2.917 | 0.000* | 0.459 | 0.512 | −1.923 | 0.024* | 0.005 |
| cg01733570 | 0.782 | 0.468 | −0.590 | 0.528 | 0.322 | 0.754 | 4.814 |
| cg03995857 | 1.453 | 0.005* | 1.462 | 0.006* | 2.465 | 0.000* | 0.679 |
| cg04317854 | 0.740 | 0.204 | 0.063 | 0.920 | 1.727 | 0.035* | 0.791 |
| cg04770504 | −0.514 | 0.461 | −0.639 | 0.298 | −1.173 | 0.039* | 1.048 |
| cg06301139 | −0.699 | 0.214 | 2.124 | 0.004* | 1.610 | 0.075 | 1.603 |
| cg07293520 | −1.598 | 0.000* | −1.525 | 0.068 | −2.353 | 0.016* | −1.803 |
| cg07320646 | 0.024 | 0.969 | −0.002 | 0.999 | 0.988 | 0.702 | −0.184 |
| cg07504598 | −0.826 | 0.130 | −0.944 | 0.286 | −2.938 | 0.206 | 1.149 |
| cg09333471 | 0.148 | 0.904 | 0.759 | 0.366 | −9.670 | 0.173 | 4.161 |
| cg12922093 | 4.882 | 0.022* | 0.220 | 0.845 | 9.063 | 0.279 | −6.404 |
| cg14205998 | −1.054 | 0.522 | −1.217 | 0.140 | 2.376 | 0.539 | 1.186 |
| cg15037663 | 0.317 | 0.792 | 0.512 | 0.617 | 1.523 | 0.545 | −2.541 |
| cg17569154 | −0.532 | 0.437 | 0.971 | 0.244 | −2.088 | 0.052 | −0.316 |
| cg17790129 | −1.223 | 0.308 | 0.836 | 0.549 | −0.585 | 0.710 | −0.227 |
| cg19260663 | −1.284 | 0.277 | −2.277 | 0.138 | 0.862 | 0.629 | −0.822 |
| cg19706795 | 1.037 | 0.137 | −0.392 | 0.575 | 0.300 | 0.741 | 0.897 |
| cg20764575 | 0.456 | 0.580 | 2.810 | 0.001* | −1.000 | 0.438 | −1.236 |
| cg22804000 | 0.329 | 0.718 | −2.476 | 0.062 | 0.568 | 0.689 | 0.853 |
| cg24805239 | 0.583 | 0.315 | −2.617 | 0.003* | 1.681 | 0.100 | −3.444 |
| cg25723149 | −1.824 | 0.002* | 0.977 | 0.366 | −0.993 | 0.216 | −2.305 |
| cg26712096 | 4.227 | 0.000* | 1.572 | 0.029* | 1.810 | 0.019* | 3.199 |

| | PAAD_p- | | PRAD_p- | | THCA_p- | | UCEC_p- |
|---|---|---|---|---|---|---|---|
| probes | value | PRAD_slope | value | THCA_slope | value | UCEC_slope | value |
| cg00473134 | 0.992 | −0.170 | 0.896 | −0.017 | 0.972 | 1.299 | 0.054 |
| cg01733570 | 0.016* | −0.083 | 0.952 | −1.810 | 0.149 | −2.340 | 0.367 |
| cg03995857 | 0.280 | 0.601 | 0.090 | 0.408 | 0.030* | −0.005 | 0.994 |
| cg04317854 | 0.174 | −0.818 | 0.081 | −0.645 | 0.283 | 0.663 | 0.618 |
| cg04770504 | 0.064 | −0.329 | 0.495 | −3.435 | 0.053 | −2.043 | 0.021* |
| cg06301139 | 0.013* | 0.652 | 0.091 | 1.764 | 0.059 | 2.265 | 0.017* |
| cg07293520 | 0.017* | −0.293 | 0.441 | 0.117 | 0.697 | −2.561 | 0.002* |
| cg07320646 | 0.830 | −0.020 | 0.954 | −2.153 | 0.137 | 0.714 | 0.691 |
| cg07504598 | 0.126 | −0.112 | 0.726 | 1.390 | 0.367 | −2.677 | 0.102 |
| cg09333471 | 0.001* | −1.840 | 0.212 | 0.251 | 0.952 | 1.412 | 0.599 |
| cg12922093 | 0.001* | −0.154 | 0.918 | −1.629 | 0.669 | 0.280 | 0.926 |
| cg14205998 | 0.265 | 0.462 | 0.305 | −3.949 | 0.185 | −4.399 | 0.035* |
| cg15037663 | 0.020* | 0.531 | 0.239 | −1.191 | 0.025* | 4.664 | 0.030* |
| cg17569154 | 0.627 | 0.083 | 0.930 | 0.396 | 0.024* | −1.683 | 0.200 |
| cg17790129 | 0.861 | −0.516 | 0.583 | −0.903 | 0.012* | −1.016 | 0.614 |
| cg19260663 | 0.508 | −1.116 | 0.241 | −0.288 | 0.382 | 2.222 | 0.350 |
| cg19706795 | 0.210 | −0.012 | 0.984 | −0.026 | 0.850 | −1.074 | 0.299 |
| cg20764575 | 0.145 | −0.812 | 0.368 | 0.105 | 0.508 | −0.648 | 0.714 |
| cg22804000 | 0.480 | −3.490 | 0.001* | −0.582 | 0.049* | −1.430 | 0.483 |
| cg24805239 | 0.001* | −0.769 | 0.429 | 0.036 | 0.891 | 2.851 | 0.023* |
| cg25723149 | 0.175 | 1.593 | 0.484 | −4.789 | 0.040* | 1.485 | 0.292 |
| cg26712096 | 0.002* | 0.930 | 0.012* | 0.987 | 0.289 | 1.683 | 0.125 |

TABLE 8
Table of the linear regression results for the analysis of age and methylation. Numbers followed by an asterisk (*) represent significant p-values.

| | | BLCA_lm_p_val- | | BRCA_lm_p_val- | | CRC_lm_p_val- | |
|---|---|---|---|---|---|---|---|
| Probe | BLCA_slope | ue | BRCA_slope | ue | CRC_slope | ue | ESCA_slope |
| cg00473134 | 0.000 | 0.449 | 0.001 | 0.006* | 0.004 | 0.000* | 0.004 |
| cg01733570 | 0.001 | 0.131 | 0.001 | 0.133 | 0.003 | 0.000* | 0.003 |
| cg03995857 | 0.001 | 0.330 | 0.002 | 0.002* | 0.004 | 0.000* | 0.005 |
| cg04317854 | −0.002 | 0.045* | 0.000 | 0.558 | 0.001 | 0.086 | 0.000 |
| cg04770504 | 0.001 | 0.279 | 0.002 | 0.000* | 0.002 | 0.004* | 0.004 |
| cg06301139 | −0.001 | 0.071 | 0.000 | 0.749 | 0.000 | 0.538 | −0.001 |
| cg07293520 | 0.000 | 0.681 | 0.001 | 0.002* | 0.005 | 0.000* | 0.005 |
| cg07320646 | 0.000 | 0.903 | 0.002 | 0.000* | 0.005 | 0.000* | 0.005 |
| cg07504598 | −0.001 | 0.133 | 0.001 | 0.093 | 0.002 | 0.002* | 0.003 |
| cg09333471 | 0.001 | 0.251 | 0.001 | 0.027* | 0.003 | 0.000* | 0.004 |
| cg12922093 | −0.002 | 0.082 | −0.001 | 0.130 | 0.000 | 0.857 | −0.002 |
| cg14205998 | −0.001 | 0.504 | −0.001 | 0.215 | 0.000 | 0.945 | −0.001 |
| cg15037663 | −0.002 | 0.010* | 0.000 | 0.531 | 0.002 | 0.005* | 0.001 |
| cg17569154 | 0.002 | 0.030* | −0.001 | 0.105 | 0.000 | 0.840 | −0.004 |
| cg17790129 | 0.000 | 0.615 | −0.001 | 0.039* | 0.000 | 0.832 | −0.002 |
| cg19260663 | 0.000 | 0.592 | −0.001 | 0.048* | 0.001 | 0.180 | −0.001 |
| cg19706795 | −0.002 | 0.011* | 0.000 | 0.942 | 0.001 | 0.122 | 0.000 |
| cg20764575 | −0.001 | 0.051 | 0.001 | 0.199 | 0.001 | 0.006* | 0.001 |
| cg22804000 | 0.000 | 0.824 | 0.001 | 0.009* | 0.002 | 0.000* | 0.004 |
| cg24805239 | 0.001 | 0.314 | 0.002 | 0.000* | 0.004 | 0.000* | 0.004 |
| cg25723149 | 0.001 | 0.419 | 0.002 | 0.000* | 0.003 | 0.000* | 0.005 |
| cg26712096 | 0.001 | 0.561 | −0.002 | 0.005* | −0.001 | 0.072 | −0.002 |

| | ESCA_lm_p_val- | | HNSC_lm_p_val- | | KIRC_lm_p_val- | | KIRP_lm_p_val- |
|---|---|---|---|---|---|---|---|
| Probe | ue | HNSC_slope | ue | KIRC_slope | ue | KIRP_slope | ue |
| cg00473134 | 0.003* | 0.000 | 0.107 | 0.000 | 0.564 | −0.002 | 0.000* |
| cg01733570 | 0.030* | 0.002 | 0.001* | 0.000 | 0.927 | −0.001 | 0.289 |
| cg03995857 | 0.002* | 0.000 | 0.065 | 0.000 | 0.745 | −0.002 | 0.000* |
| cg04317854 | 0.682 | 0.000 | 0.807 | 0.002 | 0.006* | 0.002 | 0.037* |
| cg04770504 | 0.007* | 0.000 | 0.464 | 0.000 | 0.162 | −0.001 | 0.000* |
| cg06301139 | 0.086 | 0.001 | 0.022* | 0.000 | 0.442 | 0.000 | 0.429 |
| cg07293520 | 0.004* | 0.000 | 0.909 | 0.000 | 0.644 | −0.002 | 0.000* |
| cg07320646 | 0.007* | 0.000 | 0.795 | 0.000 | 0.408 | −0.002 | 0.000* |
| cg07504598 | 0.026* | 0.002 | 0.002* | 0.000 | 0.776 | −0.002 | 0.047* |
| cg09333471 | 0.005* | 0.000 | 0.432 | 0.000 | 0.691 | −0.001 | 0.048* |
| cg12922093 | 0.012* | 0.000 | 0.115 | 0.000 | 0.405 | 0.000 | 0.756 |
| cg14205998 | 0.343 | 0.000 | 0.620 | 0.000 | 0.161 | 0.000 | 0.912 |
| cg15037663 | 0.220 | 0.002 | 0.001* | 0.000 | 0.818 | −0.002 | 0.063 |
| cg17569154 | 0.002* | 0.001 | 0.320 | 0.000 | 0.358 | −0.001 | 0.445 |
| cg17790129 | 0.016* | 0.000 | 0.154 | 0.000 | 0.545 | 0.000 | 0.543 |
| cg19260663 | 0.389 | 0.000 | 0.829 | 0.000 | 0.013* | 0.000 | 0.624 |
| cg19706795 | 0.592 | 0.002 | 0.005* | 0.000 | 0.498 | −0.002 | 0.016* |
| cg20764575 | 0.466 | 0.002 | 0.000* | 0.000 | 0.745 | −0.002 | 0.016* |
| cg22804000 | 0.001* | 0.001 | 0.001* | 0.001 | 0.158 | −0.001 | 0.110 |
| cg24805239 | 0.005* | 0.000 | 0.102 | 0.000 | 0.134 | −0.001 | 0.001* |
| cg25723149 | 0.002* | 0.002 | 0.001* | 0.001 | 0.087 | −0.001 | 0.100 |
| cg26712096 | 0.037* | 0.001 | 0.065 | 0.000 | 0.886 | 0.000 | 0.410 |

| | | LIHC_lm_p_val- | | LUAD_lm_p_val- | | LUSC_lm_p_val- | |
|---|---|---|---|---|---|---|---|
| Probe | LIHC_slope | ue | LUAD_slope | ue | LUSC_slope | ue | PAAD_slope |
| cg00473134 | 0.001 | 0.145 | 0.000 | 0.739 | 0.000 | 0.144 | 0.000 |
| cg01733570 | −0.002 | 0.026* | 0.001 | 0.284 | −0.002 | 0.045* | 0.000 |
| cg03995857 | 0.001 | 0.029* | 0.000 | 0.814 | 0.000 | 0.332 | 0.000 |
| cg04317854 | −0.001 | 0.116 | 0.001 | 0.050* | −0.001 | 0.090 | 0.001 |
| cg04770504 | 0.000 | 0.207 | 0.001 | 0.381 | 0.000 | 0.226 | 0.000 |
| cg06301139 | −0.001 | 0.134 | 0.000 | 0.726 | 0.000 | 0.450 | 0.000 |
| cg07293520 | 0.000 | 0.198 | 0.001 | 0.204 | 0.000 | 0.128 | 0.000 |
| cg07320646 | 0.000 | 0.131 | 0.001 | 0.326 | 0.000 | 0.096 | 0.000 |
| cg07504598 | −0.002 | 0.003* | 0.000 | 0.974 | −0.001 | 0.266 | 0.001 |
| cg09333471 | 0.001 | 0.041* | 0.000 | 0.656 | 0.000 | 0.752 | 0.001 |
| cg12922093 | −0.003 | 0.000* | 0.000 | 0.775 | −0.001 | 0.248 | 0.002 |
| cg14205998 | 0.000 | 0.659 | 0.000 | 0.585 | 0.000 | 0.967 | 0.000 |
| cg15037663 | −0.003 | 0.000* | 0.000 | 0.796 | −0.001 | 0.346 | 0.001 |
| cg17569154 | −0.001 | 0.036* | 0.000 | 0.502 | −0.001 | 0.278 | 0.001 |
| cg17790129 | 0.001 | 0.013* | 0.000 | 0.658 | −0.001 | 0.097 | −0.001 |
| cg19260663 | −0.003 | 0.000* | 0.001 | 0.082 | 0.000 | 0.608 | 0.001 |
| cg19706795 | −0.003 | 0.000* | 0.000 | 0.726 | −0.001 | 0.341 | 0.000 |
| cg20764575 | −0.003 | 0.000* | 0.000 | 0.944 | 0.000 | 0.897 | 0.001 |
| cg22804000 | −0.001 | 0.077 | 0.001 | 0.035* | −0.002 | 0.062 | 0.000 |
| cg24805239 | 0.000 | 0.264 | 0.001 | 0.109 | 0.000 | 0.126 | 0.001 |
| cg25723149 | −0.001 | 0.230 | 0.001 | 0.101 | −0.001 | 0.182 | 0.000 |
| cg26712096 | −0.002 | 0.004* | 0.000 | 0.497 | 0.000 | 0.911 | 0.000 |

| | PAAD_lm_p_val- | | PRAD_lm_p_val- | | THCA_lm_p_val- | | UCEC_lm_p_val- |
|---|---|---|---|---|---|---|---|
| Probe | ue | PRAD_slope | ue | THCA_slope | ue | UCEC_slope | ue |
| cg00473134 | 0.793 | 0.004 | 0.000* | 0.000 | 0.023* | 0.000 | 0.698 |
| cg01733570 | 0.814 | 0.000 | 0.949 | 0.001 | 0.033* | −0.001 | 0.218 |
| cg03995857 | 0.904 | 0.004 | 0.000* | 0.000 | 0.001* | 0.000 | 0.924 |
| cg04317854 | 0.172 | 0.001 | 0.155 | 0.001 | 0.048* | −0.001 | 0.041* |
| cg04770504 | 0.620 | 0.003 | 0.000* | 0.000 | 0.001* | 0.000 | 0.465 |
| cg06301139 | 0.396 | 0.000 | 0.276 | 0.000 | 0.125 | −0.001 | 0.112 |
| cg07293520 | 0.637 | 0.001 | 0.001* | 0.000 | 0.077 | 0.001 | 0.141 |
| cg07320646 | 0.594 | 0.001 | 0.035* | 0.000 | 0.954 | 0.001 | 0.209 |
| cg07504598 | 0.544 | 0.001 | 0.288 | 0.001 | 0.002* | 0.000 | 0.749 |
| cg09333471 | 0.116 | 0.003 | 0.004* | 0.000 | 0.239 | 0.000 | 0.883 |
| cg12922093 | 0.017* | −0.002 | 0.015* | 0.000 | 0.469 | 0.000 | 0.433 |
| cg14205998 | 0.817 | 0.000 | 0.135 | 0.000 | 0.223 | 0.000 | 0.574 |
| cg15037663 | 0.358 | 0.000 | 0.922 | 0.001 | 0.011* | 0.000 | 0.864 |
| cg17569154 | 0.208 | −0.001 | 0.217 | 0.000 | 0.567 | 0.000 | 0.781 |
| cg17790129 | 0.298 | 0.000 | 0.422 | 0.000 | 0.912 | −0.002 | 0.001* |
| cg19260663 | 0.221 | −0.002 | 0.007* | 0.000 | 0.141 | 0.000 | 0.663 |
| cg19706795 | 0.949 | 0.000 | 0.199 | 0.001 | 0.036* | 0.000 | 0.764 |
| cg20764575 | 0.200 | 0.000 | 0.454 | 0.000 | 0.282 | −0.001 | 0.131 |
| cg22804000 | 0.774 | 0.000 | 0.858 | 0.002 | 0.000* | −0.001 | 0.435 |
| cg24805239 | 0.442 | 0.004 | 0.000* | 0.001 | 0.000* | 0.000 | 0.999 |
| cg25723149 | 0.746 | 0.000 | 0.557 | 0.002 | 0.000* | −0.001 | 0.244 |
| cg26712096 | 0.315 | −0.001 | 0.223 | 0.000 | 0.413 | 0.001 | 0.154 |

### REFERENCES

1. Akino, K. et al. (2006) ‘Identification of DFNA5 as a target of epigenetic inactivation in gastric cancer’, Cancer Science, 98(1), pp. 88-95. doi: 10.1111/j.1349-7006.2006.00351.x.

2. Cohen, J. D. et al. (2018) ‘Detection and localization of surgically resecTable cancers with a multi-analyte blood test’, Science, p. eaar3247. doi: 10.1126/science.aar3247.

3. Croes, L. et al. (2017) ‘DFNA5 promoter methylation a marker for breast tumorigenesis.’, Oncotarget. Impact Journals, LLC, 8(19), pp. 31948-31958. doi: 10.18632/oncotarget.16654.

4. Croes, L. et al. (2018) ‘Large-scale analysis of DFNA5 methylation reveals its potential as biomarker for breast cancer’, Clinical Epigenetics, 10(1). doi: 10.1186/s13148-018-0479-y.

5. Ibrahim, J. et al. (2019) ‘Methylation analysis of Gasdermin E shows great promise as a biomarker for colorectal cancer’, Cancer Medicine. John Wiley & Sons, Ltd, p. cam4.2103. doi: 10.1002/cam4.2103.

6. Kim, M. S. et al. (2008) ‘Aberrant promoter methylation and tumor suppressive activity of the DFNA5 gene in colorectal carcinoma’, Oncogene, 27(25), pp. 3624-3634. doi: 10.103 8/sj.onc.1211021.

7. Kulis, M. and Esteller, M. (2010) ‘DNA Methylation and Cancer’, Advances in Genetics, 70(10), pp. 27-56. doi: 10.1016/B978-0-12-380866-0.60002-2.

8. Van Laer, L. et al. (1998) ‘Nonsyndromic hearing impairment is associated with a mutation in DFNA5.’, Nature genetics, 20(2), pp. 194-7. doi: 10.1038/2503.

9. Rogers, C. et al. (2017) ‘Cleavage of DFNA5 by caspase-3 during apoptosis mediates progression to secondary necrotic/pyroptotic cell death’, Nature Communications. Nature Publishing Group, 8, p. 14128. doi: 10.1038/ncomms14128.

10. Yokomizo, K. et al. (2012) ‘Methylation of the DFNA5 gene is frequently detected in colorectal cancer’, Anticancer Research, 32(4), pp. 1319-1322. Available at: www.ncbi.nlm.nih.gov/pubmed/22493364 (Accessed: 12 Oct. 2017).

1. A method for the ex vivo differential diagnosis between several cancer types in a subject, comprising:
a) obtaining a biological sample comprising DNA from said subject; and
b) measuring the methylation status of at least 2 CpG sites in the Gasdermin E (GSDME) gene in said biological sample,
wherein the cancer types are selected from the group consisting of bladder urothelial carcinoma, breast invasive carcinoma, oesophageal carcinoma, head and neck squamous cell carcinoma, kidney renal clear cell carcinoma, kidney renal papillary cell carcinoma, liver hepatocellular carcinoma, lung adenocarcinoma, lung squamous cell carcinoma, pancreatic adenocarcinoma, prostate adenocarcinoma, thyroid carcinoma, uterine corpus endometrial carcinoma, and colorectal carcinoma.

2. The method according to claim 1 comprising measuring the methylation status of at least 3 CpG sites in the GSDME gene in said sample.

3. The method according to claim 2 comprising measuring the methylation status of at least 6 CpG sites in the GSDME gene in said sample.

4. The method according to claim 1, wherein the at least 2 CpG sites are located in a gene body of the GSDME gene, in a putative gene promoter region of the GSDME gene, or in a region upstream of the putative gene promoter region of the GSDME gene.

5. The method according to claim 4 wherein at least 1 CpG site is located in the gene body of the GSDME gene, at least 1 CpG site is located in the putative gene promoter region of the GSDME gene, and at least 1 CpG site is located upstream of the putative gene promoter region of the GSDME gene.

6. The method according to claim 4, wherein a differential methylation status of at least 2 CpG sites in the putative gene promoter region of the GSDME gene is indicative for a differential cancer diagnosis.

7. The method according to claim 4, wherein a differential methylation status of at least 2 CpG sites in the gene body of the GSDME gene is indicative for a differential cancer diagnosis.

8. The method according claim 4, wherein a differential methylation status of at least 2 CpG sites located upstream of the putative gene promoter of the GSDME region is indicative for a differential cancer diagnosis.

9. The method according to claim 1, wherein the CpG sites are selected from the CpG sites listed in Table 1.

10. The method according to claim 3, wherein said at least 6 CpG sites are selected from CpG 3, CpG 11, CpG12, CpG13, CpG14, CpG 18, CpG19, CpG20, and CpG21 of Table 1.

11. The method according to claim 3, wherein said at least 6 CpG sites are selected from CpG 3, CpG12, CpG14, CpG18, CpG20, and CpG21 of Table 1.

12. The method according to claim 3, wherein methylation at sites CpG 3, CpG 5, CpG 6, CpG 7, CpG 19 and CpG 22 of Table 1 is indicative for bladder urothelial cancer in the subject.

13. The method according to claim 3, wherein methylation at sites CpG 2, CpG 3, CpG 4, CpG 14, CpG 17 and CpG 20 of Table 1 is indicative for breast cancer in the subject.

14. The method according to claim 3, wherein methylation at sites CpG 3, CpG 6, CpG 9, CpG 18, CpG 20 and CpG 22 of Table 1 is indicative for colorectal cancer in the subject.

15. The method according to claim 3, wherein methylation at sites CpG 1, CpG 3, CpG 7, CpG 11, CpG 14 and CpG 15 of Table 1 is indicative for esophageal cancer in the subject.

16. The method according to claims 3, wherein methylation at sites CpG 4, CpG 6, CpG 7, CpG 16, CpG 19 and CpG 20 of Table 1 is indicative for head and neck squamous cell carcinoma in the subject.

17. The method according to claim 3, wherein methylation at sites CpG 3, CpG 7, CpG 15, CpG 19, CpG 21 and CpG 22 of Table 1 is indicative for kidney renal clear cell carcinoma in the subject.

18. The method according to claim 3, wherein methylation at sites CpG 4, CpG 7, CpG 10, CpG 14, CpG 18 and CpG 22 of Table 1 is indicative for kidney renal papillary carcinoma in the subject.

19. The method according to claim 3, wherein methylation at sites CpG 3, CpG 5, CpG 6, CpG 7, CpG 13 and CpG 19 of Table 1 is indicative for liver hepatocellular carcinoma in the subject.

20. The method according to claim 3, wherein methylation at sites CpG 4, CpG 5, CpG 13, CpG 16, CpG 18 and CpG 21 of Table 1 is indicative for lung adenocarcinoma in the subject.

21. The method according to claim 3, wherein methylation at sites CpG 5, CpG 7, CpG 14, CpG 16, CpG 19 and CpG 20 of Table 1 is indicative for lung squamous cell carcinoma in the subject.

22. The method according to claim 3, wherein methylation at sites CpG 1, CpG 2, CpG 7, CpG 13, CpG 15 and CpG 22 of Table 1 is indicative for pancreatic adenocarcinoma in the subject.

23. The method according to claim 3, wherein methylation at sites CpG 1, CpG 3, CpG 10, CpG 14, CpG 16 and CpG 22 of Table 1 is indicative for prostate adenocarcinoma.

24. The method according to claim 3, wherein methylation at sites CpG 5, CpG 6, CpG 8, CpG 11, CpG 13 and CpG 21 of Table 1 is indicative for thyroid carcinoma.

25. The method according to claim 3, wherein methylation at sites CpG 1, CpG 5, CpG 14, CpG 15, CpG 16 and CpG 18 of Table 1 is indicative for uterine corpus endometrial carcinoma.

26. The method according to claim 1 wherein the methylation status of the at least 2 CpG sites, at least 3 CpG sites or at least 6 CpG sites in the GSDME gene of said subject is compared to a reference value.

27. The method according to claim 26, wherein an altered level of methylation status for said subject relative to said reference value provides an indication that the subject has cancer or provides an indication about the cancer type in said subject.

28. (canceled)

29. The method according to claim 1, wherein said biological sample is selected from the group consisting of a tissue sample, a stool sample, a cell sample, or a bodily fluid sample.

30. The method according to claim 1 wherein the DNA is DNA from liquid biopsies, circulating tumor DNA, cell-free DNA, or tumor tissue DNA.

31. (canceled)

32. The method according to claim 1 wherein the subject is a human subject.
